SEQUENCE LISTING (31 sequences)

SEQ ID NO: 1 - DNA, 25 bp, Artificial Sequence
  Primer
taggtcagttaagaacactgttctg

SEQ ID NO: 2 - DNA, 25 bp, Artificial Sequence
  Primer
tgactcatttcactaattcggtgtg

SEQ ID NO: 3 - DNA, 21 bp, Artificial Sequence
  Primer
caagcggcgactgagatgtcc

SEQ ID NO: 4 - DNA, 29 bp, Artificial Sequence
  Primer
ctgtaattttacgcatgattatctttaac

SEQ ID NO: 5 - DNA, 21 bp, Artificial Sequence
  Primer
tttgggtattgttgggaggag

SEQ ID NO: 6 - DNA, 20 bp, Artificial Sequence
  Primer
cagtttctcactacaggtac

SEQ ID NO: 7 - DNA, 19 bp, Artificial Sequence
  Primer
tgcctaagatgcccgactt

SEQ ID NO: 8 - DNA, 18 bp, Artificial Sequence
  Primer
agctgctggctggtgaag

SEQ ID NO: 9 - DNA, 22 bp, Artificial Sequence
  Primer
tgaaacctacctgatgagcaac

SEQ ID NO: 10 - DNA, 20 bp, Artificial Sequence
  Primer
agcagaacatctccgtggtt

SEQ ID NO: 11 - DNA, 21 bp, Artificial Sequence
  Primer
gcacactggtggtccatgaaa

SEQ ID NO: 12 - DNA, 20 bp, Artificial Sequence
  Primer
caagccaaacgacttccagc

SEQ ID NO: 13 - DNA, 20 bp, Artificial Sequence
  Primer
accctctgtacccccgatct

SEQ ID NO: 14 - DNA, 21 bp, Artificial Sequence
  Primer
tcgatgtagcttgctgccaac

SEQ ID NO: 15 - DNA, 20 bp, Artificial Sequence
  Primer
aatgacatcggggacaccac

SEQ ID NO: 16 - DNA, 20 bp, Artificial Sequence
  Primer
ccaccacagcatagaccgag

SEQ ID NO: 17 - DNA, 18 bp, Artificial Sequence
  Primer
gctccggtctcggagtcg

SEQ ID NO: 18 - DNA, 17 bp, Artificial Sequence
  Primer
gcgaggagcgcctagtg

SEQ ID NO: 19 - DNA, 20 bp, Artificial Sequence
  Primer
gggacaccattgccctgaaa

SEQ ID NO: 20 - DNA, 20 bp, Artificial Sequence
  Primer
gacccaacctggactcaacc

SEQ ID NO: 21 - DNA, 20 bp, Artificial Sequence
  Primer
gtccaaggcagagaaggtgc

SEQ ID NO: 22 - DNA, 20 bp, Artificial Sequence
  Primer
ctcgctggcagaaaggatga

SEQ ID NO: 23 - DNA, 19 bp, Artificial Sequence
  Primer
gggtgagcgctgagaatcg

SEQ ID NO: 24 - DNA, 16 bp, Artificial Sequence
  Primer
gcagcagggctcgcag

SEQ ID NO: 25 - DNA, 20 bp, Artificial Sequence
  Primer
gggtctgccctgagaaatcc

SEQ ID NO: 26 - DNA, 20 bp, Artificial Sequence
  Primer
tcagagcgcttgtcttgtcc

SEQ ID NO: 27 - DNA, 20 bp, Artificial Sequence
  Primer
aaactcagtgagcaaggcgt

SEQ ID NO: 28 - DNA, 22 bp, Artificial Sequence
  Primer
gggcactggtcttttccttact

SEQ ID NO: 29 - DNA, 18 bp, Artificial Sequence
  Primer
tccactggcgtcttcacc

SEQ ID NO: 30 - DNA, 21 bp, Artificial Sequence
  Primer
ggcagagatgatgaccctttt

SEQ ID NO: 31 - DNA, 22 bp, Homo sapiens
  misc_feature (1)..(22): Target sequence in human SOD1 gene
ggataacagatgagttaagggg

(19) United States
(12) Patent Application Publication
(10) Pub. No.: US 2020/0368267 A1
(43) Pub. Date: Nov. 26, 2020

(54) PROPHYLACTIC AND/OR THERAPEUTIC AGENT FOR AMYOTROPHIC LATERAL SCLEROSIS

(71) Applicant: KYOTO UNIVERSITY, Kyoto (JP)

(72) Inventors: Haruhisa INOUE, Kyoto (JP); Keiko IMAMURA, Kyoto (JP)

(73) Assignee: KYOTO UNIVERSITY, Kyoto (JP)

(21) Appl. No.: 16/766,675

(22) PCT Filed: Nov. 22, 2018
(86) PCT No.: PCT/JP2018/043242

§ 371 Date: May 22, 2020

(30) Foreign Application Priority Data
Nov. 24, 2017 (JP) 2017-226368

(51) Int. Cl. A61K 31/7088; A61P 19/04; C07K 16/18

(52) U.S. Cl. CPC A61K 31/7088; A61K 45/06; C07K 16/18; A61P 19/04

(57) ABSTRACT

The present invention provides a prophylactic and/or therapeutic agent for ALS containing a Src/c-Abl pathway inhibitor.

### TECHNICAL FIELD

The present invention relates to a prophylactic and/or therapeutic agent for amyotrophic lateral sclerosis.

### BACKGROUND ART

Amyotrophic lateral sclerosis (hereinafter also referred to as “ALS”) is a motor neuron disease of poor prognosis, which develops at middle ages and thereafter and causes progressive paralysis of skeletal muscles. It is designated as a disease in the project for investigation and research into specific diseases sponsored by the Ministry of Health, Labor and Welfare of Japan. More than about 90% of cases of ALS are sporadic and the cause is unknown. As a component of ubiquitin-positive inclusion bodies found in the lower motor neuron of sporadic ALS patients, a 43-kDa TAR DNA-binding protein (TDP-43) has been identified in recent years, and is attracting attention as an etiology gene. On the other hand, the remaining 10% are familial cases, and point mutation of genes such as Cu/Zn superoxide dismutase (SOD1) gene, TDP-43 gene and the like has been reported. In this case, the gain-of-toxic function theory is likely wherein motor neuron death is caused by the cytotoxicity newly gained by mutated SOD1.

The currently commercially available therapeutic drugs for ALS are only riluzole (Rilutek™, Aventis), which is a glutamate receptor antagonist possessing a glutamate suppressing action (patent document 1), and an antioxidant Edarabon (Radicut™, Mitsubishi Tanabe Pharma).

On the other hand, it is considered that the pathology can be reproduced in vitro by establishing an induced pluripotent stem cell (iPS cell) obtained from cells derived from a patient by using a reprogramming technique and inducing differentiation from this iPS cell into a pathogenic cell. The present inventors have demonstrated using atorvastatin known to have an anti-ALS action that a candidate substance of a therapeutic drug for ALS can be screened for by inducing differentiation of an iPS cell established from fibroblasts derived from ALS patients having a mutation in the SOD1 gene into astrocyte, and using, as an index, a decrease in the expression level of SOD1 in the obtained astrocyte (patent document 2). Furthermore, they have identified sorafenib, which is a multikinase inhibitor and used as a therapeutic drug for progressive renal cell carsinoma, as a candidate substance of a therapeutic drug for ALS, by using the screening assay (patent document 3). Also, they have shown that a candidate substance of a therapeutic drug for ALS can be screened for by inducing differentiation of iPS cell, established from ALS patients having a mutation in TDP-43 gene, into motor neuron (MN), and screening using a decrease in the expression level of TDP-43 in the obtained motor neuron, improvement of fragility to stress, recovery of neurite length and the like as indices (patent document 4, non-patent document 1). Furthermore, they have shown that a motor neuron that reproduces pathology of patients well can be promptly and synchronically prepared by introducing 3 kinds of nerve cell lineage specific transcription factors into pluripotent stem cells (patent document 5).

Thus, while a screening and efficacy evaluation system of a therapeutic drug for ALS using nerve cell derived from iPS cell is being developed, and a promising candidate substance (therapeutic drug seeds) is being found, a considerable amount of way is still expected until practicalization as a pharmaceutical product.

Incidentally, as a method for overcoming the deadlock seen recently in the development and research of a new drug a new research concept called drug repositioning (DR) has become the subject of discussion. It aims to find a new medicinal effect from existing drugs showing safety and pharmacokinetics in human, which have already been confirmed from actual results, and achieve practicalization thereof. Since many existing data can be used, it is further advantageous in that the development cost can be suppressed low, accumulated know-how and materials (surrounding compounds and the like) exist and the like. As mentioned above, the present inventors have already found that sorafenib, which is used as a therapeutic drug for cancer, has an ALS treatment activity as novel efficacy. The present inventors also found from known compound libraries that inhibitors of kinase involved in various signal transduction pathways have an ALS treatment activity (patent document 6).

However, since these kinase inhibitors are multi-kinase inhibitors having inhibitory activity against multiple kinases, it is not clear inhibition of which particular kinase or a signal transduction pathway in which the kinase is involved achieves ALS treatment activity. In addition, since these kinase inhibitors show anti-ALS activity spectra which differ depending on the causative gene of ALS, it still remains unknown which signal transduction pathway should be inhibited to achieve anti-ALS activity regardless of the causative gene.

### DOCUMENT LIST

### Patent Documents

patent document 1: AU 666150 B2

patent document 2: WO 2011/074690

patent document 3: WO 2012/029994

patent document 4: WO 2013/108926

patent document 5: WO 2014/148646

patent document 6: WO 2016/114322

### Non-Patent Document

non-patent document 1: Egawa N, et al., Sci. Transl. Med. 2012, 4: 145ra104

### SUMMARY OF THE INVENTION

### Problems to be Solved by the Invention

Therefore, the present invention aims to identify a novel drug discovery target which is truly effective for the prophylaxis and/or treatment of ALS, and capable of exhibiting a prophylactic and/or therapeutic effect for any familial or sporadic ALS regardless of the causative gene, as well as accelerate the development of a prophylactic and/or therapeutic agent for ALS as a realistic pharmaceutical product by using existing drugs effective for the target and confirmed to be safe for human.

### Means of Solving the Problems

The present inventors have induced differentiation of iPS cells established from ALS patients having a mutation in SOD1 gene into motor neuron (ALS-MN) rapidly and synchronously by the method described in the above-mentioned patent document 5, and screened for, with the survival of the motor neuron as an index, a compound having anti-ALS activity from known compound libraries including medicaments already on the market as pharmaceutical products. As a result, it was clarified that not less than half the number of hit compounds target Src/c-Abl pathway. Even when Src or c-Abl was knocked down, the survival rate of ALS-MN increased. Src/c-Abl inhibitors promoted autophagy in ALS-MN and decreased the accumulation of misfolded SOD1. Also, Src/c-Abl inhibitors improved the survival rate of motor neuron derived from the iPS cells produced from familial ALS patients with mutations in the TDP-43 gene, familial ALS patients with repeat expansion in the C9orf72 gene, and sporadic ALS patients. Furthermore, Src/c-Abl inhibitors prolonged the survival period of ALS model mice.

Based on these findings, the present inventors have concluded that Src/c-Abl pathway can be a converged treatment target widely applicable to various ALS including sporadic ALS regardless of the causative gene, and completed the present invention.

That is, the present invention provides the following.
[1] An agent for preventing and/or treating amyotrophic lateral sclerosis comprising a Src/c-Abl pathway inhibitor.
[2] The agent of [1], wherein the Src/c-Abl pathway inhibitor is selected from bosutinib, dasatinib, rebastinib, radotinib, tivozanib, pazopanib, sunitinib, BMS777607, CYC116, axitinib, KW2449, VX-680, crizotinib, MGCD-265, enzastaurin, bisindolylmaleimide I, saracatinib, imatinib, nilotinib and analogs thereof.
[3] The agent of [1], wherein the Src/c-Abl pathway inhibitor is an inhibitory nucleic acid or neutralizing antibody against Src, c-Abl, a receptor tyrosine kinase that phosphorylates Src or protein kinase C.
[4] A method for preventing and/or treating amyotrophic lateral sclerosis, comprising administering an effective amount of a Src/c-Abl pathway inhibitor to a subject in need of the prevention and/or treatment.
[5] A method for screening for a drug for preventing and/or treating amyotrophic lateral sclerosis, comprising contacting Src, c-Abl, a receptor tyrosine kinase that phosphorylates Src or protein kinase C with a test compound, and selecting a test compound that inhibits phosphorylation of the kinase as a candidate for a drug for preventing and/or treating amyotrophic lateral sclerosis.

### Effect of the Invention

According to the present invention, prophylaxis and/or treatment of various familial ALS and sporadic ALS can be performed regardless of the causative gene. In addition, since the agent for preventing and/or treating ALS of the present invention contains an existing drug with accumulated data relating to safety as the active ingredient, it is expected to shorten the development period necessary for clinical application.

### BRIEF DESCRIPTION OF THE DRAWINGS

FIG. 1 shows generation of MNs using transcription factors and modeling ALS-MNs. A. Protocol for MN generation. Scale bars, 10 μm.

B. Generated MNs present spinal MN markers HB9, ChAT, and SMI-32. Scale bars, 10 μm. C. Real-time PCR analysis shows increase in mRNA levels of HB9 and ChAT on Day 7 (each group represents mean±SEM, n=3; Student t-test, *p<0.05). D. Co-cultures with human myoblasts, Hu5/E18. Neurites of MNs co-localized with α-bungarotoxin-labeled acetylcholine receptors. Scale bar, 10 μm. E. Action potentials from current clamp recordings. F. Functional neurotransmitter receptors on generated MNs evaluated by electrophysiological analysis. Addition of 500 μM glutamate, 500 μM kainate, or 500 μM GABA induced inward currents during voltage clamp recordings. G. The percentage of HB9-positive cells on Day 7 (each group represents mean±SEM, n=3). H. Modeling ALS MNs. Misfolded SOD1 protein accumulated in MNs with mutant SOD1 gene. Scale bars, 10 μm. I. Accumulations of misfolded SOD1 protein were shown in mutant SOD1 ALS MN culture using immunoprecipitation assay. J, K. MN survival assay. Numbers of MNs on Day 7 and on Day 14 were counted by high content analysis, and the ratio of surviving MNs (Day 14/Day 7 (%)) is shown. The surviving ratio was decreased in mutant SOD1 (L144FVX) compared with control and mutation corrected clone (each group represents mean±SEM, n=6; one-way ANOVA, p<0.05, *p<0.05). Scale bars, 10 μm.

FIG. 2 shows phenotypic screening using ALS MNs and identification of therapeutic targets. A. Overview of screening flow for ALS MN survival assay. B. Through-put screening using MNs with mutant SOD1 gene (L144FVX). 1,416 compounds consisting of existing drugs and clinical trial-testing drugs were screened. Scatter plots show screening results and the highlighted compounds shown in FIG. 2D. C. Representative figures of assay results. Treatment with bosutinib increased MN survival. Scale bar, 100 μm. D. Hit drugs showed dose-dependent effects (each group represents mean±SEM, n=6; oneway ANOVA, p<0.05, *p<0.05). E. Targets of hit drugs. 14 of 27 hit drugs were included in receptor tyrosine kinase (RTK) and Src/c-Abl-associated signaling pathways. PKC; protein kinase C. F. Knock-down of Src or c-Abl increased the survival rate of mutant SOD1 ALS MNs (ALS1) (each group represents mean±SEM, n=6; one-way ANOVA, p<0.05, *p<0.05). G,H. Phosphorylation of Src/c-Abl was increased in mutant SOD1 ALS MNs, and bosutinib inhibited this phosphorylation according to western blot analysis (each group represents mean±SEM, n=3; two-way ANOVA, p<0.05, *p<0.05). I. Typical figures of immunocytostaining of p-Src/p-c-Abl in MNs. Scale bars, 10 μm. J. Increase of phosphorylation of Src/c-Abl was inhibited by treatment with bosutinib according to ELISAs (each group represents mean±SEM, n=3; two-way ANOVA, p<0.05, *p<0.05). bos; bosutinib.

FIG. 3 shows mechanistic analysis of neuroprotective effects of Src/c-Abl inhibitors on mutant SOD1 ALS MNs. A,B. Bosutinib treatment decreased the amount of p62, which was increased in mutant SOD1 MN culture, and attenuated the ratio of LC3-II/LC3-I (each group represents mean±SEM, n=3; two-way ANOVA; p<0.05, *p<0.05). C. Increase of p62 was exhibited in mutant SOD1 ALS MNs by ELISAs, and bosutinib treatment decreased the amount of p62 (each group represents mean±SEM, n=3; two-way ANOVA; p<0.05, *p<0.05). D. Rapamycin increased survival rate of mutant SOD1 ALS MNs (ALS1) (each group represents mean±SEM, n=6; one-way ANOVA, p<0.05, *p<0.05). E. Knock-down of mTOR increased survival rate of mutant SOD1 ALS MNs (ALS1) (each group represents mean±SEM, n=6; Student t-test, p<0.05). F. Autophagy inhibitors, LY294002 and chloroquine, decreased the protective effect of bosutinib on MN survival assay (each group represents mean±SEM, n=6; two-way ANOVA, p<0.05, *p<0.05). G, H. Immunoprecipitation analysis (G) and ELISA (H) showed that bosutinib treatment decreased the misfolded SOD1 protein level, which was elevated in mutant SOD1 MN culture. I. Bosutinib treatment did not decrease SOD1 mRNA expression level. J. Intracellular ATP level was decreased in mutant SOD1 MN culture. Bosutinib to partially attenuated the ATP shortage (each group represents mean±SEM, n=6, Two-way ANOVA; p<0.05, *p<0.05). K. Gene Set Enrichment Analysis of single-cell RNA sequencing showed up-regulation for genes in TCA cycle and respiratory electron transport (control 1; n=10, control 2; n=11, ALS1; n=23, ALS3; n=21). bos; bosutinib.

FIG. 4 shows effect of Src/c-Abl inhibitor on iPSC-derived MNs with different genotypes and on ALS model mice. A. iPSC-derived MNs of each clone on Day 7. Scale bars, 100 μm. B. Bosutinib increased MN survival of mutant TDP-43-, and C9orf72-repeat expansion mediated familial ALS and from a part of sporadic ALS (each group represents mean±SEM, n=6; one-way ANOVA, p<0.05; post hoc test, p<0.05). C. Kaplan-Meier analysis showed that bosutinib delayed disease onset of mutant SOD1 Tg mice (bosutinib; 123.2±9.1 days, vehicle; 112.4±14.4 days, mean±SD, log-rank test, p=0.0021, n=26 per group). D. Kaplan-Meier analysis showed that bosutinib extended the survival time of mutant SOD1 Tg mice (bosutinib; 164.1±9.4 days, vehicle; 156.3±8.5 days, mean±SD, log rank test, p=0.0019, n=26 per group). E. Misfolded SOD1 protein in spinal cord at 12 weeks of age was evaluated by ELISA. Bosutinib decreased the misfolded SOD1 accumulations in spinal cord (each group represents mean±SEM, non-transgenic littermates (non-Tg); n=3, Tg treated with vehicle; n=3, Tg treated with bosutinib; n=3, one-way ANOVA, p<0.05; post hoc test, p<0.05). F. Typical image of Cresyl violet-stained section of ventral horn from the lumbar spinal cord at the late symptomatic stage. Scale bars, 50 μm. G. The number of MNs on one side of the lumbar spinal cord was quantified (each group represents mean±SEM, non-Tg; n=4, Tg treated with vehicle; n=5, Tg treated with bosutinib; n=5, one-way ANOVA; p<0.05, *p<0.05). bos; bosutinib.

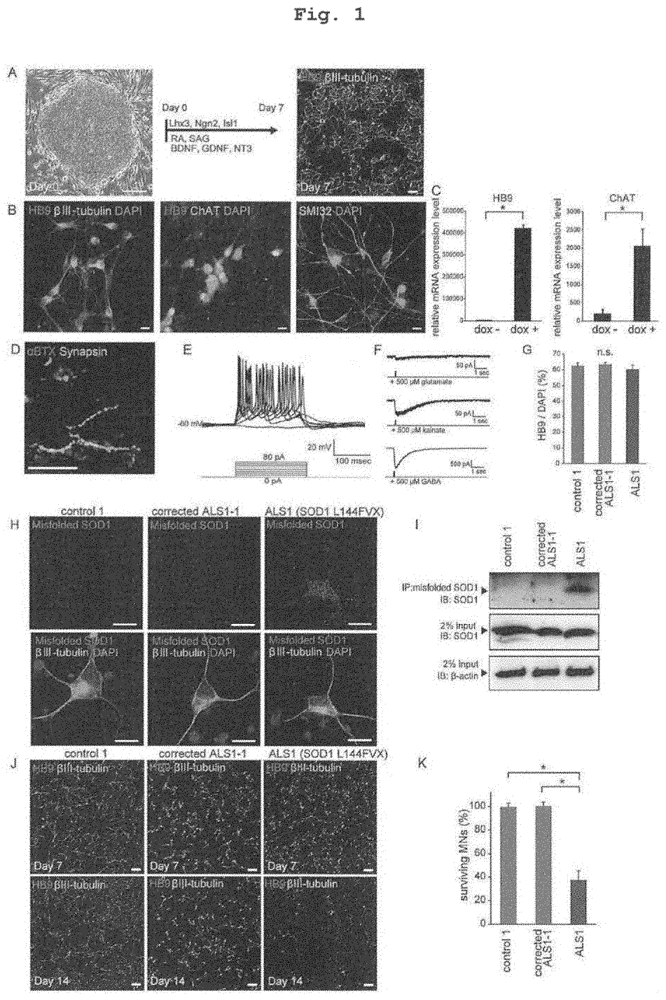

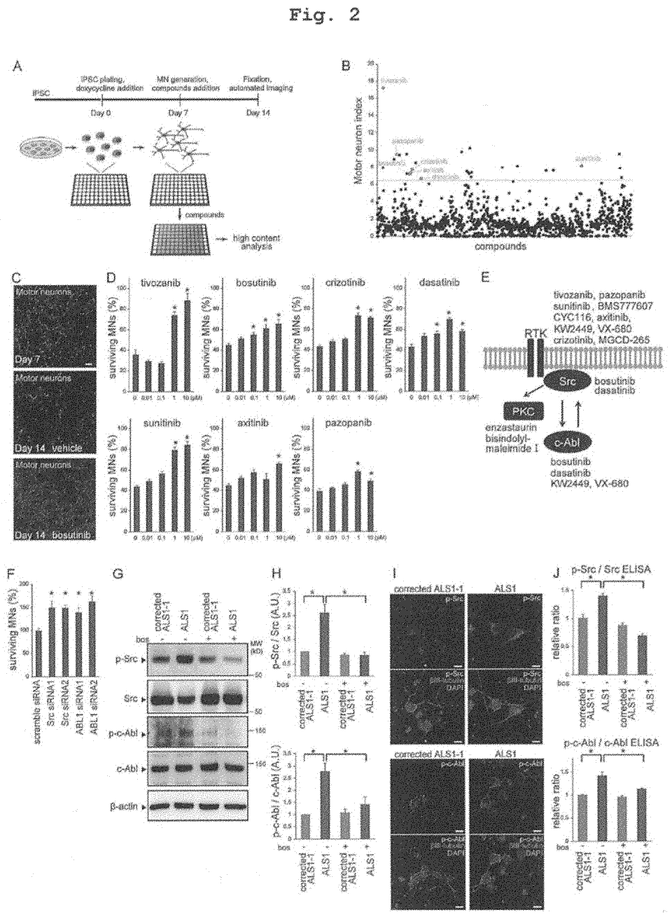

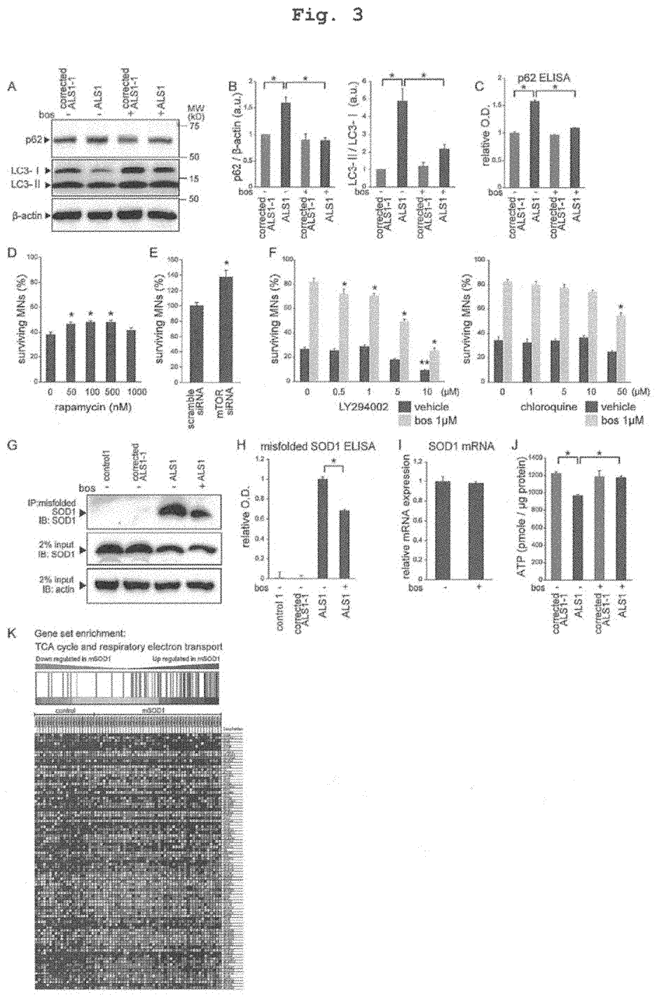

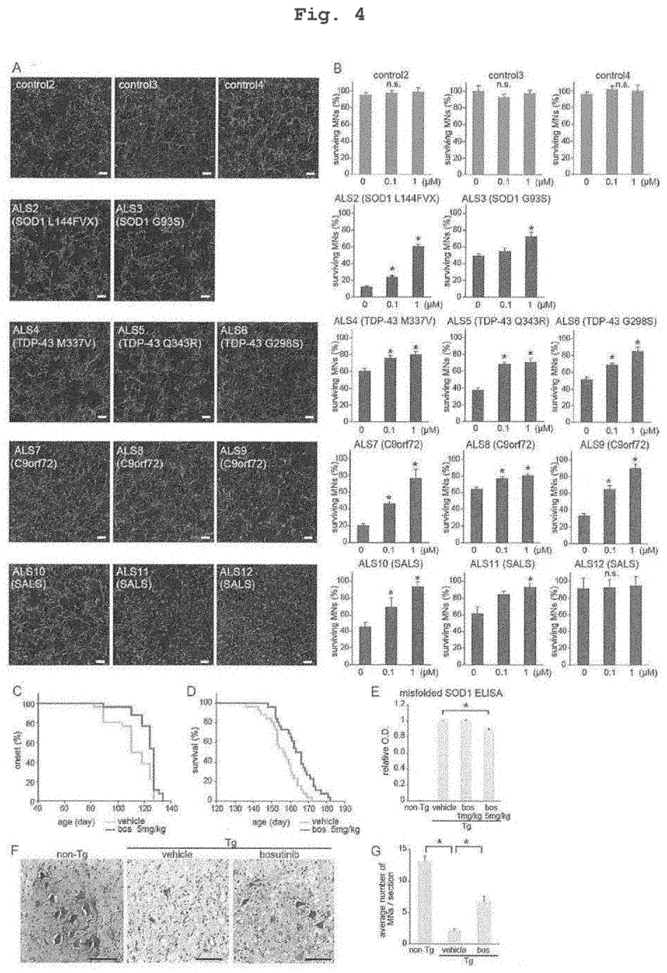

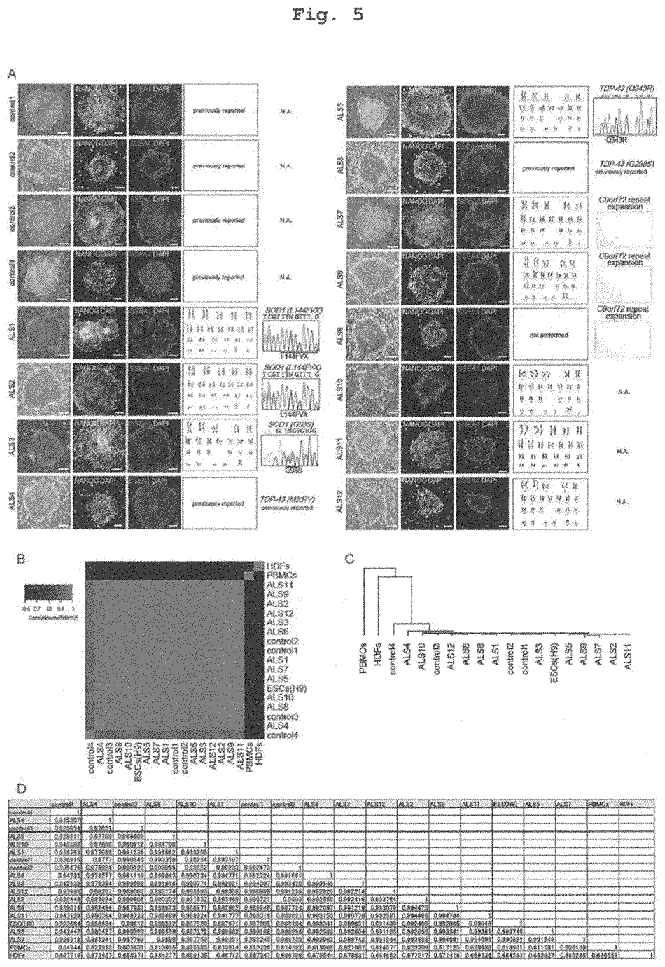

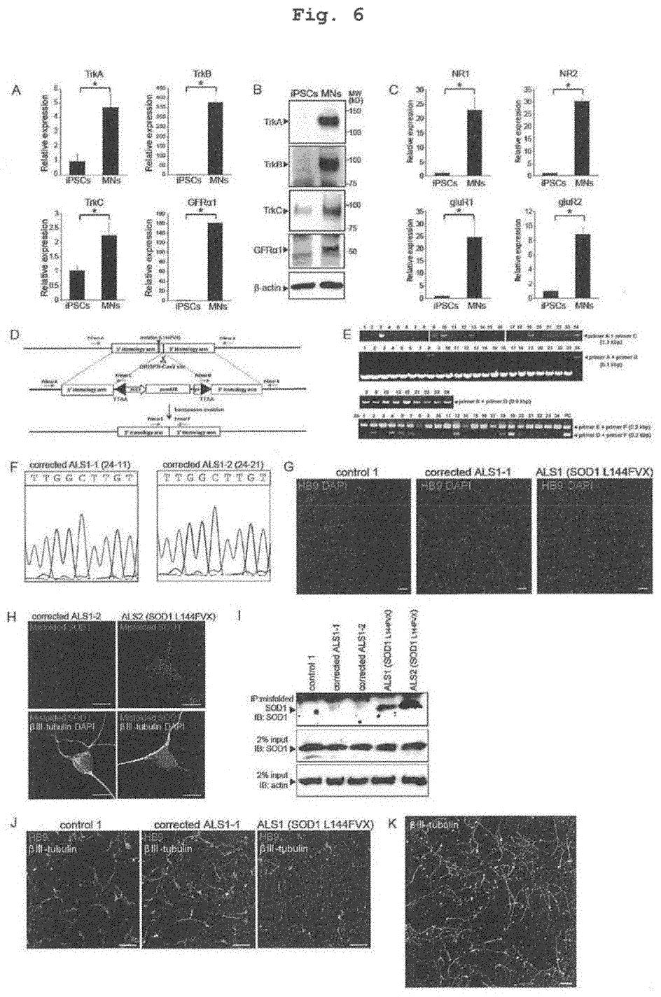

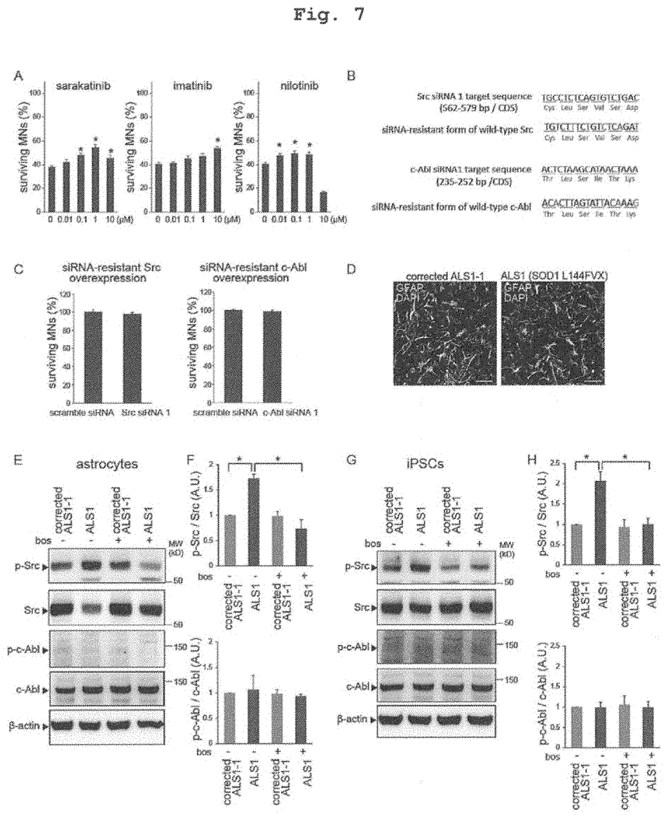

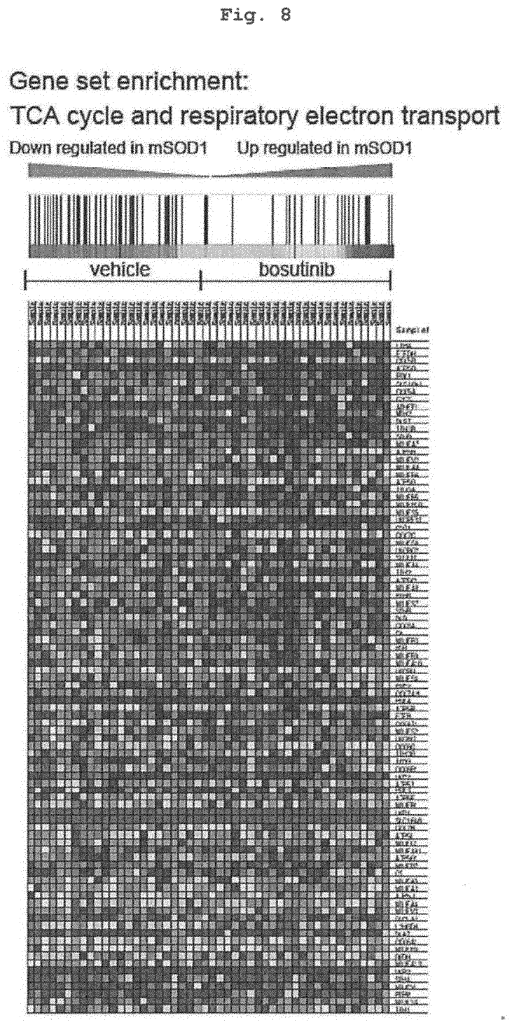

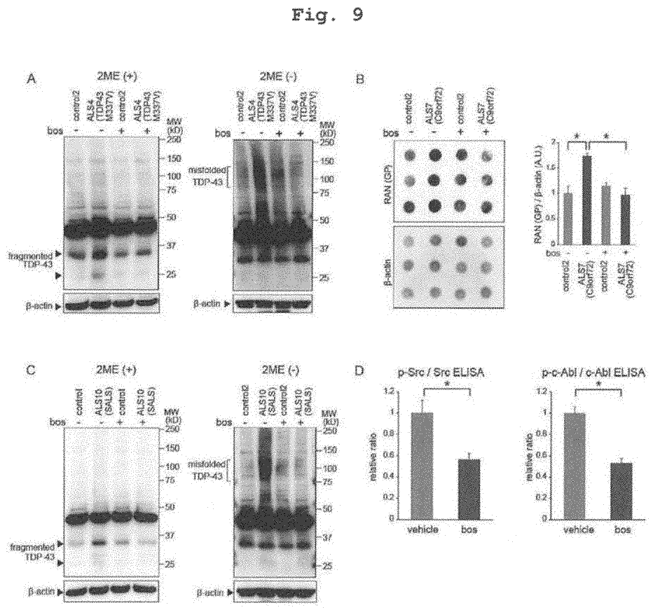

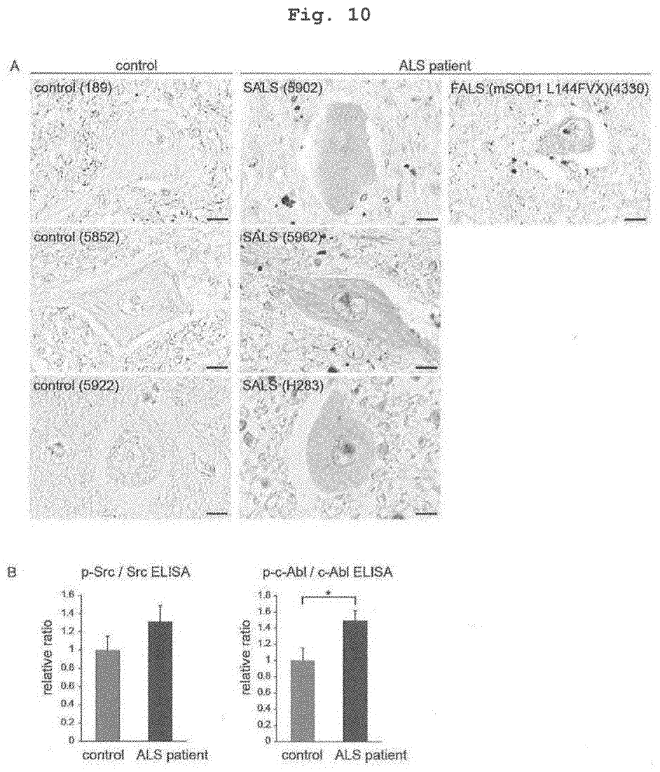

FIG. 5 shows control and production of iPS cells derived from ALS patients. A. iPS cells were produced from familial ALS patients with mutations in the TDP-43 gene, familial ALS patients with mutations in the SOD1 gene, familial ALS patients with repeat expansion in the C9orf72 gene, and sporadic ALS patients. The produced iPS cells showed embryonic stem cell (ESC)-like morphology (pase contrast image) and expressed pluripotent stem cell markers NANOG and SSEA4. The scale bar is 100 μm. By karyotype analysis, it was clarified that the produced iPS cells maintain a normal karyotype. B. Comprehensive gene expression analysis of iPS cell, ESC (H9), HDF and PBMC. The Pearson's correlation coefficient was calculated for the paired samples of 19 cell line. The values are indicated by the upper left color scale. C. Hierarchical clustering of RMA-summarized microarray data was performed using the mean concatenation method and the distance metric based on Pearson's correlation coefficient. D. Pearson's correlation coefficient of each comprehensive gene profile. FIG. 6 shows characterization of MN for ALS-MN modeling and gene repair of SOD1 mutant iPS cells. A. The produced MN expressed mRNAs of TrkA, TrkB, TrkC and GFRal (in each group, mean±SEM, n=3; Student's t-test, * p<0.05). B. Western blotting revealed that the produced MN expressed TrkA, TrkB, TrkC and GFRα1 proteins. C. The produced MN expressed mRNAs of NR1, NR2, gluR1 and gluR2 (in each group, mean±SEM, n=3; Student's t-test, *p<0.05). D. Using CRISPR-Cas9 system, mutation (SOD1 L144FVX) in ALS1 was repaired (repaired ALS1-1, repaired ALS1-2). CRISPR-Cas9 produced a double-stranded cleavage and induced homologous recombination with a targeting plasmid containing the repaired sequence and the puromycin resistance cassette. After successful targetting, the puromycin resistance cassette was removed by transient expression of piggyBac transposase. E. PCR genotyping to accurately select targeted clones. F. Mutation repair after gene editing was clarified by Sanger sequence analysis. G. Immunostaining for HB9 and DAPI on day 7. The scale bar is 100 μm. H and I. Accumulation of misfolded SOD1 was evaluated. Consistent with FIG. 1H, I, accumulation of misfolded SOD1 protein in ALS2 MN derived from different patients with mutations in the SOD1 gene is clear. However, the second clone with the repaired SOD1 mutation (repaired ALS1-2) did not show misfolded SOD1. The scale bar is 10 μm. J. Almost 100% of βIII-tubulin positive neuron expressed HB9. The scale bar is 100 μm. K. Raw data of MN survival test. Neurites (red) and cell bodies (blue) were detected by optimized fluorescence levels. The number of cell bodies having neurites was defined as the number of surviving nerves. The scale bar is 100 μm.

FIG. 7 shows investigation of the effects of Src/c-Abl inhibitors. A. Sarakatinib, imatinib and nilotinib as other Src/c-Abl inhibitors increased MN survival (in each group, mean±SEM, n=6; one-way analysis of variance: p<0.05, *; p<0.05). B. Wild-type Src (top) or wild-type c-Abl (bottom) siRNA resistant form was introduced into iPS cells. C. wild-type Src and wild-type c-Abl siRNA resistance form inhibited the effects of Src siRNA and c-Abl siRNA (in each group, mean±SEM, n=6, n.s.). D. Immunostaining for differentiation of iPS cell-derived astrocytes on day 100. The scale bar is 100 μm. E and F. Phosphorylation of Src increased on day 100 in SOD1-mutant ALS astrocytes, and bosutinib inhibited phosphorylation (in each group, mean±SEM, n=3; 2-way factorial analysis of variance: p<0.05, *p<0.05). Phosphorylation of c-Abl did not increase in SOD1-mutant ALS astrocytes (n=3). G and H. Phosphorylation of Src increased in SOD1-mutant ALS iPS cells, and bosutinib inhibited phosphorylation (in each group, mean±SEM, n=3; 2-way analysis of variance: p<0.05, *p<0.05). Phosphorylation of c-Abl did not increase in SOD1-mutant ALS iPS cells (n=3).

FIG. 8 shows Changes in mRNA expression by bosutinib treatment in single cell analysis. Single cell analysis was performed on ALS1 MN with or without bosinib treatment for 72 hr. mRNA expression was associated with the TCA cycle and electron transport in MN treated with bosutinib decreased compared to untreated MN (GSEA: FDR=0.07, vehicle-treated ALS1: n=23; bosutinib-treated ALS1: n=25).

FIG. 9 shows decrease of misfolded proteins by treatment with bosutinib. A. Western blotting showed that bostinib decreased fragmented TDP-43 in mutant TDP-43-related ALS MN under reducing conditions, and decreased misfolded TDP-43 in mutant TDP-43-related ALS MN under non-reducing conditions. B. Dot blot analysis showed that bosutinib decreased RAN (anti-GP repeat antibody) in ALS MN associated with elongation of C9orf72 repeat sequences (in each group, mean±SEM, n=3; 2-way factorial analysis of variance: p<0.05, *p<0.05). C. Western blotting showed that bostinib decreased fragmented TDP-43 in sporadic ALS MN under reducing conditions and decreased misfolded TDP-43 in sporadic ALS MN under non-reducing conditions. D. Bosutinib inhibited Src/c-Abl in the spinal cord of SOD1-mutant Tg mice (in each group, mean±SEM, n=3; Student's t-test, *; p<0.05).

FIG. 10 shows investigation of the spinal cord of ALS patients. A. Phosphorylated Src immunoreactivity increased in MN in the spinal cord of ALS patients. The scale bar is 10 μm. B. Phosphorylation of Src/c-Abl was investigated by ELISA using human spinal cord samples. Phosphorylation of Src in the spinal cord of ALS patients showed an increasing tendency, although there was no statistically significant difference compared with the control. Phosphorylation of c-Abl in the spinal cord of ALS patients significantly increased compared with the control. In each group, mean±SEM, control n=12, ALS patients n=9; Student's t-test, *; p<0.05.

### DESCRIPTION OF EMBODIMENTS

The present invention provides an agent for preventing and/or treating amyotrophic lateral sclerosis (ALS) containing a Src/c-Abl pathway inhibitor (hereinafter to be also referred to as “the prophylactic/therapeutic agent of the present invention”).

In the present invention, ALS to be the treatment target encompasses both sporadic ALS and familial ALS. In the case of familial ALS, the causative gene is not particularly limited, and may be any known causative gene such as SOD1, TDP-43, C9orf72, alsin, SETX, FUS/TLS, VAPB, ANG, FIG4, OPTN, ATXN2, DAO, UBQLN2, PFN1, DCTN1, CHPM2B, VCP and the like. In one embodiment, in the case of familial ALS having SOD1 mutation, examples of the SOD1 gene mutation include, but are not limited to, a mutation in which the 144th Leu of SOD1 protein is substituted by Phe-Val-Xaa (Xaa is any amino acid) (SOD1-L144FVX), a mutation in which the 93rd Gly is substituted by Ser (SOD1-G93S), a mutation in which the 106th Leu is substituted by Val (SOD1-L106V) and the like. In another embodiment, in the case of familial ALS having TDP-43 mutation, examples of the TDP-43 gene mutation include, but are not limited to, a mutation in which the 337th Met of TDP-43 protein is substituted by Val (TDP-43-M337V), a mutation in which the 343rd Gln is substituted by Arg (TDP-43-Q343R), a mutation in which the 298th Gly is substituted by Ser (TDP-43-G298S) and the like. In still another embodiment, in the case of familial ALS having C9orf72 mutation, examples of the C9orf72 SOD1 gene mutation include, but are not limited to, (GGGGCC)n repeats of abnormal elongation in intron 1.

In the present specification, the “Src/c-Abl pathway” means a signal transduction pathway involving Src, c-Abl and a receptor tyrosine kinase (RTK) that phosphorylates Src and protein kinase C (PKC) as substrates, which is shown in FIG. 2E (in the Figure, arrows show phosphorylation reactions). The Src/c-Abl pathway inhibitor which is the active ingredient of the prophylactic/therapeutic agent of the present invention includes all of naturally-occurring substances derived from microorganisms, semi-synthetic substances derived therefrom, and totally synthetic compounds, as long as they inhibit the activity and/or expression of at least one of the above-mentioned kinases constituting the Src/c-Abl pathway.

Examples of the Src/c-Abl pathway inhibitor include low-molecular-weight compounds such as bosutinib, dasatinib, rebastinib, radotinib, tivozanib, pazopanib, sunitinib, BMS777607, CYC116, axitinib, KW2449, VX-680, crizotinib, MGCD-265, enzastaurin, bisindolylmaleimide I, sarakatinib, imatinib, nilotinib and analogs thereof and the like. Among these, bosutinib, dasatinib and rebastinib inhibit both Src and c-Abl, radotinib, tivozanib, pazopanib, sunitinib, BMS777607, CYC116, axitinib, KW2449, VX-680, crizotinib and MGCD-265 inhibit RTK, and enzastaurin and bisindolylmaleimide I inhibits PKC.

Examples of the bosutinib analogs, tivozanib analogs, pazopanib analogs, sunitinib analogs, axitinib analogs and crizotinib analogs include the compounds described in the above-mentioned patent document 6.

The imatinib analog is, for example, a compound represented by the following formula (I):

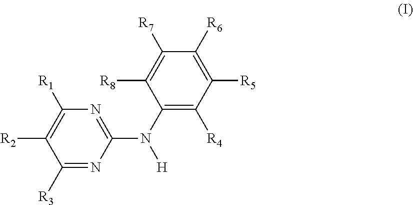

[wherein:

R₁ is 4-pyrazinyl, 1-methyl-1H-pyrrolyl, amino- or amino-lower alkyl-substituted phenyl [amino group in each case is free, or alkylated or acylated], 1H-indolyl or 1H-imidazolyl bonded via a carbon atom of a 5-membered ring, or unsubstituted or lower alkyl-substituted pyridyl which is bonded via a carbon atom of the ring and substituted or not substituted with oxygen at nitrogen atom, R₂ and R₃ are each independently hydrogen or lower alkyl, 1 or 2 groups of groups R₄, R₅, R₆, R₇ and R₈ is/are each nitro, fluoro-substituted lower alkoxy, or a group of the following formula (II)

—N(R₉)—C(═X)—(Y)ₙ—R₁₀ (II)

[wherein R₉ is hydrogen or lower alkyl, X is oxo, thio, imino, N-lower alkyl-imino, hydroxyimino or O-lower alkyl-hydroxyimino, Y is oxygen or group NH, n is 0 or 1, R₁₀ is aliphatic group having at least 5 carbon atoms, or aromatic, aromatic-aliphatic, alicyclic, alicyclic-aliphatic, heterocyclic, or heterocyclic-aliphatic group], and the remaining groups R₄, R₅, R₆, R₇ and R₈ are each independently hydrogen, lower alkyl unsubstituted, free, or substituted by alkylated amino, piperazinyl, piperidinyl, pyrrolidinyl or morpholinyl, or lower alkanoyl, trifluoromethyl, or free, etherified or esterified hydroxy, or free, alkylated or acylated amino, or free or esterified carboxy].

Each term described as higher concept in the explanation of each group in the above-mentioned formula (II) (e.g., “lower alkyl”, “aliphatic group having at least 5 carbon atoms” etc.), and other terms follow the definitions in JP-A-6-087834.

Specific examples of the analog of imatinib include the following compounds.

N-(3-nitrophenyl)-4-(3-pyridyl)-2-pyrimidine-amine;

N-[3-(4-chloro benzoylamide)-phenyl]-4-(3-pyridyl)-2-pyrimidine-amine; N-(3-benzoylamide-phenyl)-4-(3-pyridyl)-2-pyrimidine-amine;

N-[3-(2-pyridyl)carboxamide-phenyl]-4-(3-pyridyl)-2-pyrimidine-amine;

N-[3-(3-pyridyl)carboxamide-phenyl]-4-(3-pyridyl)-2-pyrimidine-amine;

N-[3-(4-pyridyl)carboxamide-phenyl]-4-(3-pyridyl)-2-pyrimidine-amine;

N-(3-penta fluoro-benzoylamide-phenyl)-4-(3-pyridyl)-2-pyrimidine-amine;

N-[3-(2-carboxy-benzoylamide)phenyl]-4-(3-pyridyl)-2-pyrimidine-amine;

N-(3-n-hexanoylamide-phenyl)-4-(3-pyridyl)-2-pyrimidine-amine;

N-(3-nitro-phenyl)-4-(2-pyridyl)-2-pyrimidine-amine;

N-(3-nitro-phenyl)-4-(4-pyridyl)-2-pyrimidine-amine;

N-[3-(2-methoxy-benzoylamide)-phenyl]-4-(3-pyridyl)-2-pyrimidine-amine;

N-[3-(4-fluoro-benzoylamide)-phenyl]-4-(3-pyridyl)-2-pyrimidine-amine;

N-[3-(4-cyano-benzoylamide)-phenyl]-4-(3-pyridyl)-2-pyrimidine-amine;

N-[3-(2-thienylcarboxamide)-phenyl]-4-(3-pyridyl)-2-pyrimidine-amine;

N-(3-cyclohexylcarboxamide-phenyl)-4-(3-pyridyl)-2-pydine-amine;

N-[3-(4-methyl-benzoylamide)-phenyl]-4-(3-pyridyl)-2-pyrimidine-amine;

N-[3-(4-chloro benzoylamide)-phenyl]-4-(4-pyridyl)-2-pyrimidine-amine;

N-{3-[4-(4-methyl-piperazinomethyl)-benzoylamide]-phenyl}-4-(3-pyridyl)-2-pyrimidine-amine;

N-(5-benzoylamide-2-methyl-phenyl)-4-(3-pyridyl)-2-pyrimidine-amine;

N-{5-[4-(4-methyl-piperazino-methyl)-benzoylamide]-2-methyl-phenyl}-4-(3-pyridyl)-2-pyrimidine-amine;

N-[5-(4-methyl-benzoylamide)-2-methylphenyl]-4-(3-pyridyl)-2-pyrimidine-amine;

N-[5-(2-naphthoylamide)-2-methyl-phenyl]-4-(3-pyridyl)-2-pyrimidine-amine;

N-[5-(4-chloro-benzoylamide)-2-methyl-phenyl]-4-(3-pyridyl)-2-pyrimidine-amine;

N-[5-(2-methoxy-benzoylamide)-2-methyl-phenyl]-4-(3-pyridyl)-2-pyrimidine-amine;

N-(3-trifluoro-methoxy-phenyl)-4-(3-pyridyl)-2-pyrimidine-amine;

N-(3-[1,1,2,2-tetra fluoro-ethoxy]-phenyl)-4-(3-pyridyl)-2-pyrimidine-amine;

N-(3-nitro-5-methyl-phenyl)-4-(3-pyridyl)-2-pyrimidine-amine;

N-(3-nitro-5-trufluoro methyl-phenyl)-4-(3-pyridyl)-2-pyrimidine-amine;

N-(3-nitro-phenyl)-4-(N-oxide-3-pyridyl)-2-pyrimidine-amine;

N-(3-benzoyl-amide-5-methyl-phenyl)-4-(N-oxide-3-pyridyl)-2-pyrimidine-amine.

The nilotinib analog is, for example, a compound represented by the following formula:

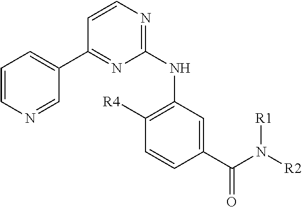

[wherein:

R₁ is hydrogen, lower alkyl, lower alkoxy-lower alkyl, acyloxy-lower alkyl, carboxy-lower alkyl, lower alkoxycarbonyl-lower alkyl or phenyl-lower alkyl;

R₂ is hydrogen, lower alkyl optionally substituted by one or more, the same or different residues R₃ when desired, cycloalkyl, benzcycloalkyl, heterocyclyl, aryl group, or a monocyclic or bicyclic heteroaryl group having 0, 1, 2, or 3 ring nitrogen atoms and 0 or 1 ring oxygen atom and 0 or 1 ring sulfur atom, wherein each group is unsubstituted or mono- or poly-substituted; and

R₃ is hydroxy, lower alkoxy, acyloxy, carboxy, lower alkoxycarbonyl, carbamoyl, N-mono- or N,N-di-substituted carbamoyl, amino, mono- or di-substituted amino, cycloalkyl, heterocyclyl, aryl group, or monocyclic or bicyclic heteroaryl group having 0, 1, 2, or 3 ring nitrogen atoms and 0 or 1 ring oxygen atom and 0 or 1 ring sulfur atom, wherein each group is unsubstituted or mono- or poly-substituted; or

R₁ and R₂ are joined to show alkylene having 4, 5 or 6 carbon atoms and optionally mono- or di-substituted by lower alkyl, cycloalkyl, heterocyclyl, phenyl, hydroxy, lower alkoxy, amino, mono- or di-substituted amino, oxo, pyridyl, pyrazinyl or pyrimidinyl when desired; benz alkylene having 4 or 5 carbon atoms; oxa alkylene having one oxygen and 3 or 4 carbon atoms; or azaalkylene having one nitrogen and 3 or 4 carbon atoms and having nitrogen unsubstituted or substituted by lower alkyl, phenyl-lower alkyl, lower alkoxycarbonyl-lower alkyl, carboxy-lower alkyl, carbamoyl lower alkyl, N-mono- or N,N-di-substituted carbamoyl lower alkyl, cycloalkyl, lower alkoxycarbonyl, carboxy, phenyl, substituted phenyl, pyridinyl, pyrimidinyl or pyrazinyl; and

R₄ is hydrogen, lower alkyl or halogen].

Each term described as higher concept in the explanation of each group in the above-mentioned formula (IV) (e.g., “lower alkyl”, “halogen” etc.), and other terms follow the definitions in WO 2004/005281 (JP-A-2005-533827).

Specific examples of the analog of nilotinib include the following compounds.

4-methyl-3-[[4-(3-pyridinyl)-2-pyrimidinyl]amino]benzamide;

4-methyl-3-[[4-(3-pyridinyl)-2-pyrimidinyl]]aminobenzanilide;

4-methyl-N-(3-pyridinyl)-3-[[4-(3-pyridinyl)-2-pyrimidinyl]amino]benzamide;

N-(4-chlorophenyl)-4-methyl-3-[[4-(3-pyridinyl)-2-pyrimidinyl]amino]benzamide;

2(R)- and 2(S)-[4-methyl-3-[[4-(3-pyridinyl)-2-pyrimidinyl]amino]benzoylamino]propanoic acid;

4-methyl-3-[[4-(3-pyridinyl)-2-pyrimidinyl] amino]-N-(8-quinolinyl)benzamide;

4-methyl-3-[[4-(3-pyridinyl)-2-pyrimidinyl]amino]-N-(3-[trifluoromethoxy]phenyl)benzamide;

4-methyl-3-[[4-(3-pyridinyl)-2-pyrimidinyl]amino]-N-(2-pyrrolidinoethyl)benzamide;

4-methyl-3-[[4-(3-pyridinyl)-2-pyrimidinyl]amino]-N-(3-pyrrolidinophenyl)benzamide;

4-methyl-3-[[4-(3-pyridinyl)-2-pyrimidinyl]amino]-N-(1-[2-pyrimidinyl]-4-piperidinyl])benzamide;

N-(4-di[2-methoxyethyl]amino-3-trifluoromethylphenyl)-4-methyl-3-[[4-(3-pyridinyl)-2-pyrimidinyl]amino]benzamide;

N-(4-[1H-imidazolyl]-3-trifluoromethylphenyl])-4-methyl-3-[[4-(3-pyridinyl)-2-pyrimidinyl]amino]benzamide;

4-methyl-3-[[4-(3-pyridinyl)-2-pyrimidinyl]amino]-N-(2-pyrrolidino-5-trif N-(3,4-difluoro phenyl)-4-methyl-3-[[4-(3-pyridinyl)-2- pyrimidinyl]amino]benzamide;

4-methyl-3-[[4-(3-pyridinyl)-2-pyrimidinyl]amino]-N-(3-trifluoromethylphenyl)benzamide;

N-(3-chloro-5-trifluoromethylphenyl)-4-methyl-3-[[4-(3-pyridinyl)-2-pyrimidinyl]amino]benzamide;

N-(4-diethylaminobutyl)-4-methyl-3-[[4-(3-pyridinyl)-2-pyrimidinyl]amino]benzamide;

4-methyl-N-[4-(4-methyl-1-piperazinyl)-3-trifluoromethylphenyl]-3-[[4-(3-pyridinyl)-2-pyrimidinyl]amino]benzamide;

4-methyl-3-[[4-(3-pyridinyl)-2-pyrimidinyl]amino]-N-[4-(2,2,2-trifluoroethoxy)-3-trifluoromethylphenyl]benzamide;

4-methyl-N-[4-(2-methyl-1H-imidazolyl)-3-trifluoromethylphenyl]-3-[[4-(3-pyridinyl)-2-pyrimidinyl]amino]benzamide;

4-methyl-N-(4-phenyl-3-trifluoromethylphenyl)-3-[[4-(3-pyridinyl)-2-pyrimidinyl]amino]benzamide;

4-methyl-N-[4-(4-methyl-1H-imidazolyl)-3-trifluoromethylphenyl]-3-[[4-(3-pyridinyl)-2-pyrimidinyl]amino]benzamide;

methyl 2(R)- and 2(S)-[4-methyl-3-[[4-(3-pyridinyl)-2-pyrimidinyl]amino]benzoylamino]-3-[4-hydroxyphenyl]propanoate;

N-[2-(N-cyclohexyl-N-methylaminomethyl)phenyl]-4-methyl-3-[[4-(3-pyridinyl)-2-pyrimidinyl]amino]benzamide;

N-[3-[2-(1H-imidazolyl)ethoxy]phenyl]-4-methyl-3-[[4-(3-30 pyridinyl)-2-pyrimidinyl]amino]benzamide;

4-methyl-N-[3-morpholino-5-trifluoromethylphenyl]-3-[[4-(3-pyridinyl)-2-pyrimidinyl]amino]benzamide;

4-methyl-3-[[4-(3-pyridinyl)4-methyl-3-[[4-(3-pyridinyl)-2-pyrimidinyl]amino]benzamide;

N-(4-diethylamino-3-trifluoromethylphenyl)-4-methyl-3-[[4-(3-pyridinyl)-2-pyrimidinyl]amino]benzamide;

4-methyl-3-[[4-(3-pyridinyl)-2-pyrimidinyl]amino]-N-[3-(3-pyridinyl)-5-trifluorophenyl]benzamide;

N-[3-[3-(1H-1-imidazolyl)propoxy]phenyl]-4-methyl-3-[[4-(3-5 pyridinyl)-2-pyrimidinyl]amino]benzamide;

4-methyl-3-[[4-(3-pyridinyl)-2-pyrimidinyl]amino]-N-[4-(3-pyridinyl)-3-trifluorophenyl]benzamide;

4-methyl-N-[3-(4-methyl-l-piperazinyl)-5-trif1uorophenyl]-3-[[4-(3-pyridinyl)-2-pyrimidinyl]amino]benzamide;

4-methyl-N-[3-methylcarbamoyl-5-trifluorophenyl]-3-[[4-(3-pyridinyl)-2-pyrimidinyl]amino]benzamide;

4-methyl-N-[3-methylcarbamoyl-5-morpholino]-3-[[4-(3-pyridinyl)-2-pyrimidinyl]amino]benzamide;

4-methyl-3-[[4-(3-pyridinyl)-2-pyrimidinyl]amino]-N-[3-[3-(1H-imidazol-1-yl)propoxy]phenyl]benzamide;

4-methyl-3-[[4-(3-pyridinyl)-2-pyrimidinyl]amino]-N-[3-[2-(1H-imidazol-1-yl)ethoxy]phenyl]benzamide;

4-methyl-3-[[4-(3-pyridinyl)-2-pyrimidinyl]amino]-N-[4-(ethylamino)-3-(trifluoromethyl)phenyl]benzamide;

4-methyl-3-[[4-(3-pyridinyl)-2-pyrimidinyl]amino]-N-[-(diethylamino)-3-(trifluoromethyl)phenyl]benzamide;

(±)-4-methyl-3-[[4-(3-pyridinyl)-2-pyrimidinyl]amino]-N-[4-[(2-hydroxypropyl)amino]-3-(trifluoromethyl)phenyl]benzamide;

4-methyl-3-[[4-(3-pyridinyl)-2-pyrimidinyl]amino]-N-[4-[bis (2-methoxyethyl)amino]-3-(trifluoromethyl)phenyl]benzamide;

4-methyl-3-[[4-(3-pyridinyl)-2-pyrimidinyl]amino]-N-[4-(4-methyl-1-piperazinyl)-3-(trifluoromethyl)phenyl]benzamide;

4-methyl-3-[[4-(3-pyridinyl)-2-pyrimidinyl]amino]-N-[4-(1-piperidinyl)-3-(trifluoromethyl)phenyl]benzamide;

4-methyl-3-[[4-(3-pyridinyl)-2-pyrimidinyl]amino]-N-[4-(1-pyrrolidinyl)-3-(trifluoromethyl)phenyl]benzamide;

4-methyl-3-[[4-(3-pyridinyl)-2-pyrimidinyl]amino]-N-[4-(4-morpholinyl)-3-(trifluoromethyl)phenyl]benzamide;

4-methyl-3-[[4-(3-pyridinyl)-2-pyrimidinyl]amino]-N-[4-phenyl-3-(trifluoromethyl)phenyl]benzamide;

4-methyl-3-[[4-(3-pyridinyl)-2-pyrimidinyl]amino]-N-[3-[4-(3-pyridinyl)-3-(trifluoromethyl)phenyl]methyl]benzamide;

4-methyl-3-[[4-(3-pyridinyl)-2-pyrimidinyl]amino]-N-[4-(1H-imidazol-1-yl)-3-(trifluoromethyl)phenyl]benzamide;

4-methyl-3-[[4-(3-pyridinyl)-2-pyrimidinyl]amino]-N-[4-(2,4-dimethyl-1H-imidazol-1-yl)-3-(trifluoromethyl)phenyl]benzamide;

4-methyl-3-[[4-(3-pyridinyl)-2-pyrimidinyl]amino]-N-[4-(4-methyl-1H-imidazol-1-yl)-3-(trifluoromethyl)phenyl]benzamide;

4-methyl-3-[[4-(3-pyridinyl)-2-pyrimidinyl]amino]-N-[4-(2-methyl-1H-imidazol-1-yl)-3-(trifluoromethyl)phenyl]benzamide;

4-methyl-3-[[4-(3-pyridinyl)-2-pyrimidinyl]amino]-N-[3-(4-morpholinyl)-5-[(methylamino)carbonyl]phenyl]benzamide;

4-methyl-3-[[4-(3-pyridinyl)-2-pyrimidinyl]amino]-N-[3-[(methylamino)carbonyl]-5-(trifluoromethyl)phenyl]benzamide;

4-methyl-3-[[4-(3-pyridinyl)-2-pyrimidinyl]amino]-N-[5-(3-pyridinyl)-3-(trifluoromethyl)phenyl]benzamide;

4-methyl-3-[[4-(3-pyridinyl)-2-pyrimidinyl]amino]-N-[5-(4-morpholinyl)-3-(trifluoromethyl)phenyl]benzamide;

4-methyl-3-[[4-(3-pyridinyl)-2-pyrimidinyl]amino]-N-[5-(2-20 methyl-1H-imidazol-1-yl)-3-(trifluoromethyl)phenyl]benzamide;

4-methyl-3-[[4-(3-pyridinyl)-2-pyrimidinyl]amino]-N-[5-(4-methyl-1H-imidazol-1-yl)-3-(trifluoromethyl)phenyl]benzamide;

4-methyl-3-[[4-(3-pyridinyl)-2-pyrimidinyl]amino]-N-[5-(5-methyl-1H-imidazol-1-yl)-3-(trifluoromethyl)phenyl]benzamide;

4-methyl-3-[[4-(3-pyridinyl)-2-pyrimidinyl]amino]-N-[3-(4-methyl-1-piperazinyl) -5-(trifluoromethyl)phenyl]benzamide;

4-methyl-3-[[4-(3-pyridinyl)-2-pyrimidinyl]amino]-N-[2-(1-pyrrolidinyl)-5-(trifluoromethyl)phenyl]benzamide

As the above-mentioned low-molecular-weight Src/c-Abl pathway inhibitor, a commercially available product may be used, or each compound can be produced by a method known per se.

For example, bosutinib or an analog thereof can be produced according to the methods described in, for example, U.S. Pat. Nos. 6,002,008 and 6,780,996, or Boschelli, D. H. et al., J. Med. Chem., 44, 3965 (2001), Boschelli, D. H. et al., J Med. Chem., 44, 822 (2001), Boschelli, D. H. et al., Bioorg. Med. Chem. Lett., 13, 3797 (2003), Boschelli, D. H. et al., J. Med. Chem., 47, 1599 (2004), and Ye, F. et al., 221th National Meeting of the American Chemical Society, San diego, California (April, 2001).

Tivozanib or an analog thereof can be produced according to the method described in, for example, WO 02/088110.

Pazopanib or an analog thereof can be produced according to the method described in, for example, WO 2002/059110 (JP-A-2004-517925).

Crizotinib or an analog thereof can be produced according to the method described in, for example, WO 2004/076412 (JP-A-2006-519232).

Sunitinib or an analog thereof can be produced according to the method described in, for example, WO 01/060814 (JP-A-2003-523340).

Axitinib or an analog thereof can be produced according to the method described in, for example, WO 01/002369 (JP-A-2003-503481).

Imatinib or an analog thereof can be produced according to the method described in, for example, JP-A-H06-087834).

Nilotinib or an analog thereof can be produced according to the method described in, for example, WO 2004/005281 (JP-A-2005-533827).

The Src/c-Abl way inhibitor encompasses not only a free form but also a pharmacologically acceptable salt thereof. While the pharmacologically acceptable salt varies depending on the kind of the compound, examples thereof include base addition salts such as salts with inorganic base such as alkali metal salts (sodium salt, potassium salt etc.), alkaline earth metal salts (calcium salt, magnesium salt etc.), aluminum salt, ammonium salt and the like, and salts with organic base such as trimethylamine, triethylamine, pyridine, picoline, ethanolamine, diethanolamine, triethanolamine, dicyclohexylamine, N,N′-dibenzylethylenediamine and the like and the like, and acid addition salts such as salts with inorganic acid such as hydrochloride, hydrobromide, sulfate, hydroiodide, nitrate salt, phosphate and the like, and salts with organic acid such as citrate, oxalate, acetate, formate, propionate, benzoate, trifluoroacetate, maleate, tartrate, methanesulfonate, benzenesulfonate, paratoluenesulfonate and the like, and the like.

When the Src/c-Abl pathway inhibitor contains isomers such as an optical isomer, a stereoisomer, a regioisomer or a rotamer, any one of the isomers and mixtures are also encompassed in the Src/c-Abl pathway inhibitor of the present invention. For example, when the Src/c-Abl pathway inhibitor of the present invention contains an optical isomer, an optical isomer resolved from racemate is also encompassed in the Src/c-Abl pathway inhibitor of the present invention.

These isomers can be obtained as single products by a synthesis method, a separation method (e.g., concentration, solvent extraction, column chromatography, recrystallization etc.), an optical resolution method (e.g., fractional recrystallization, chiral column method, diastereomer method etc.) and the like each known per se.

The Src/c-Abl pathway inhibitor of the present invention may be a crystal, and is included in the compound of the present invention whether it is in a single crystal form or a crystal mixture. The crystal can be produced by crystallizing by applying a crystallization method known per se.

The Src/c-Abl pathway inhibitor of the present invention may be a solvate (e.g., hydrate etc.) or a non-solvate (e.g., non-hydrate etc.), both of which are encompassed in the compound of the present invention.

In addition, a compound labeled with an isotope (e.g., ³H, ¹⁴C, ³⁵S, ¹²⁵I etc.) is also encompassed in the Src/c-Abl pathway inhibitor of the present invention.

In another embodiment, examples of the Src/c-Abl pathway inhibitor include kinases constituting the Src/c-Abl pathway, i.e., inhibitory nucleic acids such as antisense nucleic acid, siRNA, shRNA, miRNA, ribozyme and the like against Src, c-Abl, RTK that phosphorylates Src or PKC as substrates. These inhibitory nucleic acids can be appropriately designed using design software known per se based on the nucleotide sequence information of the gene encoding each kinase that constitutes the Src/c-Abl pathway, and easily synthesized using an automatic DNA/RNA synthesizer. Some of these inhibitory nucleic acids are commercially available, and they can also be used. For example, Src siRNA (siRNA ID:s13414, s13413 etc.), c-Abl siRNA (siRNA ID:s866, s864 etc.) and the like are available from Life Technologies.

In another embodiment, the Src/c-Abl pathway inhibitor may be a neutralizing antibody against kinase constituting the Src/c-Abl pathway. Particularly, an antibody which is a transmembrane protein that specifically recognizes the extracellular domain of RTK that phosphorylates Src as a substrate, and inhibits phosphorylation of Src by RTK can be mentioned as a preferable example, but it is not limited thereto. These antibodies can be appropriately produced using a method known per se, or commercially available antibodies can also be used.

The prophylactic and/or therapeutic agent of the present invention can be administered orally or parenterally in the form of the active ingredient the compound of the present invention as it is alone, or as a pharmaceutical composition in an appropriate dosage form blended with a pharmacologically acceptable carrier, excipient, diluent and the like.

As the composition for oral administration, solid or liquid dosage forms, specifically tablets (including sugar-coated tablets and film-coated tablets), pills, granules, powders, capsules (including soft capsules), syrups, emulsions, suspensions and the like can be mentioned. Meanwhile, as examples of the composition for parenteral administration, injections, suppositories and the like are used; the injections may include dosage forms such as intravenous injections, subcutaneous injections, intracutaneous injections, intramuscular injections and drip infusion injections. These preparations are produced by a well-known method using additives, including excipients (e.g., organic excipients like sugar derivatives such as lactose, sucrose, glucose, mannitol, and sorbitol; starch derivatives such as cornstarch, potato starch, a starch, and dextrin; cellulose derivatives such as crystalline cellulose; gum arabic; dextran; and pullulan; and inorganic excipients like silicate derivatives such as light anhydrous silicic acid, synthetic aluminum silicate, calcium silicate, and magnesium metasilicoaluminate; phosphates such as calcium hydrogen phosphate; carbonates such as calcium carbonate; and sulfates such as calcium sulfate), lubricants (e.g., stearic acid, stearic acid metal salts such as calcium stearate and magnesium stearate; talc; colloidal silica; waxes such as beeswax and spermaceti; boric acid; adipic acid; sulfates such as sodium sulfate; glycol; fumaric acid; sodium benzoate; DL leucine; lauryl sulfates such as sodium lauryl sulfate and magnesium lauryl sulfate; silicates such as silicic anhydride and silicic hydrates; and the aforementioned starch derivatives), binders (e.g., hydroxypropylcellulose, hydroxypropylmethylcellulose, polyvinylpyrrolidone, macrogol, and the same compounds as the aforementioned excipients), disintegrants (e.g., cellulose derivatives such as low-substitutional hydroxypropylcellulose, carboxymethylcellulose, carboxymethylcellulose calcium, and internally crosslinked carboxymethylcellulose sodium; chemically modified starches and celluloses such as carboxymethylstarch, carboxymethylstarch sodium, and crosslinked polyvinylpyrrolidone), emulsifiers (e.g., colloidal clays such as bentonite and Veegum; metal hydroxides such as magnesium hydroxide and aluminum hydroxide; anionic surfactants such as sodium lauryl sulfate and calcium stearate; cationic surfactants such as benzalkonium chloride; and non-ionic surfactants such as polyoxyethylene alkyl ethers, polyoxyethylene sorbitan fatty acid ester of, and sucrose fatty acid ester), stabilizers (para-oxybenzoic acid esters such as methyl paraben and propyl paraben; alcohols such as chlorobutanol, benzyl alcohol, and phenylethyl alcohol; benzalkonium chloride; phenols such as phenol and cresol; thimerosal; dehydroacetic acid; and sorbic acid), taste/odor correctives (e.g., sweeteners, souring agents, and flavors in common use), and diluents.

The dose of the Src/c-Abl pathway inhibitor which is the active ingredient of the prophylactic and/or therapeutic agent in the present invention is variable according to various conditions such as the kind of compound, the patient's symptoms, age, weight, drug acceptability and the like. At least 0.1 mg (suitably 0.5 mg) to at most 1000 mg (suitably 500 mg) per dose for oral administration, or at least 0.01 mg (suitably 0.05 mg) to at most 100 mg (suitably 50 mg) per dose for parenteral administration, can be administered to an adult 1 to 6 times a day. The dose may be increased or reduced according to the symptoms. Particularly, when the Src/c-Abl pathway inhibitor is already on the market as a pharmaceutical product for a disease other than the above-mentioned diseases, an appropriate dose of each compound can be determined within the range confirmed to be safe.

Furthermore, the prophylactic and/or therapeutic agent for ALS of the present invention may be used in combination with other drugs, for example, glutamic acid action suppressants (e.g., riluzole and the like), antioxidant (e.g., Edaravone etc.), neurotrophic factors [e.g., insulin-like growth factor-1, 5-HT_{1A} receptor agonists (e.g., xaliproden) and the like] and the like. The prophylactic and/or therapeutic agent of the present invention and these other drugs can be administered simultaneously, sequentially, or separately.

The present invention also provides a method for screening for a prophylactic and/or therapeutic drug for ALS, including contacting a kinase constituting Src/c-Abl pathway (i.e., Src, c-Abl, RTK that phosphorylates Src or PKC) with a test compound, and selecting a test compound that inhibits phosphorylation of the kinase as a candidate for a prophylactic and/or therapeutic drug for ALS (hereinafter to be also referred to as “the screening method of the present invention”).

For example, in one embodiment, the screening method of the present invention includes
(1) a step of contacting mammalian cells having Src/c-Abl pathway with a test compound,
(2) a step of measuring the level of phosphorylation of any kinase constituting the Src/c-Abl pathway,
(3) a step of comparing the level of phosphorylation in the above-mentioned (2) with the level of phosphorylation of the kinase in the cell not contacted with the test compound, and
(4) a step of selecting a test compound that decreased the level of phosphorylation of the kinase as a candidate for a prophylactic and/or therapeutic agent for ALS.

The mammalian cell to be used in step (1) is not particularly limited. ALS-MN may also be used or may be a cell line frequently used typically. For example, to facilitate the measurement later, a cell incorporating constitutively active RTK gene can also be used.

The test compound to be used in the screening method of the present invention is not particularly limited, and protein, peptide, nucleic acid, inorganic compound, natural or synthetically prepared organic compound and the like can be mentioned. Specific examples of the test compound include peptide libraries of 3 to 50, preferably 5 to 20, amino acid residues, and libraries of low-molecular-weight organic compounds having a molecular weight of 100 to 2000, preferably 200 to 800, prepared using a combinatorial chemistry technique known to those skilled in the art.

The concentration of the test compound to be in contact with the cells is not particularly limited, and may be generally about 0.01 μM to about 100 μM, preferably 0.1 μM to 50 μM. The time for contacting the cells with the test compound is not particularly limited and may be determined as appropriate. It is, for example, about 5 min to 30 min, preferably about 10 min to 20 min. The test compound can be used by appropriately dissolving or suspending in water, a buffer such as a phosphate buffer or a Tris buffer, a solvent such as ethanol, acetone, dimethyl sulfoxide, or a mixture thereof.

The degree of phosphorylation in step (2) can be determined by, for example, measuring the amount of phosphorylated kinase before and after contact with the test compound (or in comparison to control cells) by using antibodies specific for each phosphorylated kinase protein and by Western blotting, pull-down assay, or other immunological methods. As preferable embodiments, for example, analysis using a confocal laser microscope in fluorescence immunostaining, flow cytometry using fluorescence antibody and the like can be mentioned.

When the degree of phosphorylation of the kinase protein in the cells added with the test compound is statistically significantly reduced as compared to the degree of phosphorylation in the control cells free of addition of the test compound, the test compound can be selected as a candidate for a drug for preventing and/or treating ALS.

The present invention is explained in more detail in the following by referring to Examples, which are not to be construed as limitative.

### EXAMPLE

### Materials and Methods

### Study Design

Using the cell phenotype of motor neuron (ALS-MN) induced from iPS cells derived from familial and sporadic patients by the method described in the above-mentioned patent document 5, throughput drug screening was performed. Of the hit drugs, candidate targets for ALS treatment were focused on and verified using plural ALS iPS cell clones. The in vivo effect was analyzed using ALS model mice. The production and use of human iPS cells were approved by the ethics committees of each department including Kyoto University. All methods were performed according to the approved guidelines. Formal informed consent was obtained from all test subjects. All mice analyzed in this Example were controlled and the procedure was performed according to the guidelines of the Kyoto University Animal Research Institute, and all experiments were performed with the approval of the CiRA Animal Care and Use Committee. Human post-mortem samples with written informed consent were obtained from School of Medicine and Graduate School of Medicine, Kyoto University, Jichi Medical University, and Kansai Medical University.

### Production of iPS Cells

iPS cells were produced using retrovirus (Sox2, Klf4, Oct3/4 and c-Myc), sendaivirus (Sox2, Klf4, Oct3/4 and c-Myc), or episomal vector (Sox2, Klf4, Oct3/4, L-Myc, Lin28 and p53-shRNA) from skin fibroblasts, peripheral blood mononuclear cells (PBMC) or immortalized B lymphocytes [Cell 131, 861-872 (2007); Stem Cells 31, 458-466 (2013); Proc Jpn Acad Ser B Phys Biol Sci 85, 348-362 (2009)], and cultured on an SNL feeder layer using human iPS cell medium (primates embryonic stem cell medium; ReproCELL, Yokohama, Japan) supplemented with 4 ng/ml bFGF (Wako Pure Chemical Industries, Ltd., Osaka, Japan) and penicillin/streptomycin.

### Microarray Analysis

Using RNeasy Plus Mini Kit (QIAGEN), total RNA of iPS cells was extracted. cDNA was synthesized from the total RNA (200 ng) using GeneChip WT PLUS Reagent Kit (Affymetrix), and the obtained cDNA was fragmented and hybridized to Human Gene 2.0 ST Array (Affymetrix). After hybridization, GeneChip array was washed, stained with GeneChip Fluidics Station 450 (Affymetrix), and detected by Scanner 3000 TG system (Affymetrix) according to the standard protocol of the manufacturer. The data analysis was performed using GeneSpringGX 12.6 (Agilent Technologies) software and R-software.

### Chemical Substance for Screening

Tivozanib and crizotinib were purchased from LKT Laboratories (St. Paul, Min.), bosutinib from Abcam (Cambridge, UK), sunitinib from SIGMA (St. Louis, Mo.), axitinib, pazopanib and saracatinib from Selleck Chemicals (Houston, Tex.), dasatinib from Santa Crus Biotechnology (Dallas, Tex.), and kenpaullone from Tocris (Missouri, UK).

### Production of ALS iPS Cell Clone Relating to Isogenic Mutant SOD1 Using CRISPR-Cas9 Gene Editing Technique

To repair mutation of SOD1 gene by CRISPR-Cas9, a guide RNA targetting 5′-GGATAACAGATGAGTTAAGGGG-3′ (SEQ ID NO: 31) site was designed using CRISPR Design (http://crispr.mit.edu/). To express guide RNA oligonucleotide from human H1 polymerase III promoter, the guide RNA was inserted into the BamHI-EcoRI site in pHL-H1-ccdB-EF1α-RiH plasmid (Li, H.L., et al., Stem Cell Reports 4, 143-154 (2015)). To construct a donor plasmid, 5′ and 3′ homology arms having a normal SOD1 gene sequence, and PuroΔLTK cassette flanked by piggyBac terminal repeats were inserted into pBluescript SK(+). For CRISPR-Cas9 transfection, 10 μg of pHL-H1 guide RNA expression plasmid, 10 μg of pHL-EF1α-hcSpCas9 plasmid, and 10 μg of donor plasmid were electroporated into one million iPS cells by a NEPA21 electroporator (Nepagene). Four days after transfection, puromycin selection was performed for 10 days. Puromycin resistant colonies were randomly selected and expanded for genomic DNA extraction and genotyping by PCR with primers A, B, C and D (shown in Table 1). The amplified PCR band was further analyzed by Sanger sequencing to confirm the expected repair and lack of sequence change in the homology arms. To remove the PuroiTK cassette, piggyBac transposase that expresses vector pHL-EFla-hcPBase-A (Matsui, H., et al., PLoS One 9, e104957 (2014)) was electroporated into the target clone, and removal of the puromycin cassette was confirmed by PCR using primers D, E and F (Table 1).

TABLE 1
List of primers for SOD1 gene editing

| primer | | SEQ ID |
|---|---|---|
| list | sequence (5′ → 3′) | NO: |
| primer A | TAGGTCAGTTAAGAACACTGTTCTG | 1 |
| primer B | TGACTCATTTCACTAATTCGGTGTG | 2 |
| primer C | CAAGCGGCGACTGAGATGTCC | 3 |
| primer D | CTGTAATTTTACGCATGATTATCTTTAAC | 4 |
| primer E | TTTGGGTATTGTTGGGAGGAG | 5 |
| primer F | CAGTTTCTCACTACAGGTAC | 6 |

### Production of Polycistronic Vector and Introduction Into iPS Cells

A polycistronic vector containing mouse Lhx3, mouse Ngn2 and mouse Is11 under the control of a tetracycline operator and a minimum CMV promoter was produced from KW111_PB_TAC_ERN having rtTA and neomycin resistance gene (Efla_rtTA_neo) (used in the experiments of FIGS. 1-3 and FIGS. 6-8) and KW110_PB_TA_ERN (Ef1a_rtTA_neo) vector backbone (used in the experiments of FIG. 4 and FIG. 9) [Kim, S. I., et al., Methods Mol Biol 1357, 111-31 (2015)]. Lhx3, Ngn2 and Isl1 were purchased from Origene and linked using two F2A sequences (LNI cassette). Then, the produced vector was co-transfected into iPS cells together with pCyL43 vector encoding transposase by using lipofectamine LTX (Thermo Fisher Scientific). After clone selection using neomycin, iPS cells having tetracycline-inducing LNI cassette were established.

### Production of Motor Neuron (MN)

iPS cells with LNI cassette introduced therein were dissociated into single cells using Accutase (Innovative Cell Technologies), seeded on a dish coated with Matrigel or cover glass, and cultured for 7 days together with 1 μg/ml of oxycycline (TAKARA, Kusatsu, Japan) in N3 medium (DMEM/F12 (Thermo Fisher Scientific), 100 μg/ml apotransferrin (Sigma), 5 μg/ml insulin (Sigma), 30 nM selenious acid (Sigma), 20 nM progesterone (Sigma), and 100 nM putrescine (Sigma)) containing 1 μM retinoic acid (Sigma), 1 μM Smoothened Agonist (SAG), 10 ng/ml BDNF (R&D Systems), 10 ng/ml GDNF (R&D Systems) and 10 ng/ml NT-3 (R&D Systems).

### Quantitative RT-PCR

Total RNA of cultured cells was extracted using RNeasy Plus Mini kit (QIAGEN). Using 1 microgram of RNA was reverse transcribed using ReverTra Ace (TOYOBO). Quantitative PCR analysis was performed by reverse transcription reaction with SYBR Premix ExTaqII (TAKARA) using StepOnePlus (Thermo Fisher Scientific). The primer sets are listed in Table 2.

TABLE 2
List of primers for qPCR

| primer | | SEQ ID |
|---|---|---|
| list | sequence (5′ → 3′) | NO: |
| HB9_F | TGCCTAAGATGCCCGACTT | 7 |
| HB9_R | AGCTGCTGGCTGGTGAAG | 8 |
| ChAT_F | TGAAACCTACCTGATGAGCAAC | 9 |
| ChAT_R | AGCAGAACATCTCCGTGGTT | 10 |
| SOD1_F | GCACACTGGTGGTCCATGAAA | 11 |
| SOD1_R | CAAGCCAAACGACTTCCAGC | 12 |
| TrkA_F | ACCCTCTGTACCCCCGATCT | 13 |
| TrkA_R | TCGATGTAGCTTGCTGCCAAC | 14 |
| TrkB-F | AATGACATCGGGGACACCAC | 15 |
| TrkB-R | CCACCACAGCATAGACCGAG | 16 |
| TrkC-F | GCTCCGGTCTCGGAGTCG | 17 |
| TrkC-R | GCGAGGAGCGCCTAGTG | 18 |
| GFRα1_F | GGGACACCATTGCCCTGAAA | 19 |
| GFRα1_R | GACCCAACCTGGACTCAACC | 20 |
| NR1_F | GTCCAAGGCAGAGAAGGTGC | 21 |
| NR1_R | CTCGCTGGCAGAAAGGATGA | 22 |
| NR2_F | GGGTGAGCGCTGAGAATCG | 23 |
| NR2_R | GCAGCAGGGCTCGCAG | 24 |
| GluR1_F | GGGTCTGCCCTGAGAAATCC | 25 |
| GluR1_R | TCAGAGCGCTTGTCTTGTCC | 26 |
| GluR2_F | AAACTCAGTGAGCAAGGCGT | 27 |
| GluR2_R | GGGCACTGGTCTTTTCCTTACT | 28 |
| GAPDH_F | TCCACTGGCGTCTTCACC | 29 |
| GAPDH_R | GGCAGAGATGATGACCCTTTT | 30 |

### Immunocytochemistry

Cells were fixed with 4% paraformaldehyde at room temperature for 30 min, washed with PBS, and permeabilized in PBS containing 0.2% Triton X-100 for 10 min at room temperature, followed by blocking with Block Ace (Yukijirushi) for 30 min. After incubating at 4° C. overnight with primary antibody, the cells were washed 3 times with PBS and then incubated with the appropriate secondary antibody for 1 hr at room temperature. Using Delta Vision (GE Healthcare), BIOREVO (Keyence) or IN Cell Analyzer 6000 (GE Healthcare), cell images were acquired. The cell number was quantified using IN Cell Analyzer 6000 and IN Cell Developer toolbox software 1.9 (GE Healthcare). In this assay, the following primary antibodies were used: βIII-tubulin (1:2,000, Covance; 1:1,000, Abcam), HB9 (1:200, Developmental Studies Hybridoma Bank), ChAT (1:100, Millipore), GFAP (1:500, Santa Cruz), Ibal (1:1,000, Wako), misfolded SOD1 (B8H10) (1:100, MEDIMABS), Nestin (1:200, Millipore), Nanog (1:700, ReproCELL), SSEA-4 (1:200, Millipore), phosphorylated Src (1:100, R&D Systems) and phosphorylated c-Abl (1:100, SAB, College Park, Md.).

### Electrophysiological Recording

Whole cell patch clamp recording was performed using iPS cell-derived MN under a microscope in combination with differential interference contrast imaging method. A recording micropipette was filled with an intracellular solution composed of 140 mM KCl, 2 mM MgCl₂, 10 mM HEPES and 1 mM EGTA and adjusted to pH 7.4 with NaOH. During the experiment, the cells were maintained at 30° C., and continuously perfused with oxygenated Krebs-Ringer solution composed of 125 mM NaCl, 2.5 mM KCl, 1.25 mM NaH₂ PO₄, 26 mM NaHCO₃, 1 mM MgCl₂, 2 mM CaCl₂, and 20 mM glucose. Voltage and current clamp recordings were made using EPC9 amplifier (HEKA) and the data were analyzed using Patchmaster software (HEKA). For testing functional neurotransmitter receptors, as described above (Miles, G. B., et al., J Neurosci 24, 7848-7858 (2004)), 500 μM glutamate and 500 μM GABA were ejected using a pneumatic PicoPump (World Precision Instruments) through a micropipette with a tip diameter of 2-3 μm at a low pressure (less than 10 psi) for 50 ms and placed about 5 μm away from the cell body.

### Western Blot

The cells were recovered and lysed in RIPA buffer containing 0.1% SDS, 150 mM NaCl, 1% NP-40, 0.5% deoxycholate, 50 mM Tris-HCl (pH 8.0), protease inhibitor (Roche) and phosphatase inhibitor (Roche). The sample was centrifuged at 13,000×g for 15 min at 4° C. The concentration of protein in the supernatant was measured using a bicinchoninic acid (BCA) assay kit (Thermo Fisher Scientific). The total protein extract (10 μg/lane) was separated by size on a 10-20% polyacrylamide gel and transferred to Immobilon-P membrane (Millipore). The membrane was blocked by Blocking One (Nacalai Tesque), hybridized with an appropriate antibody and visualized using ECL Prime detection kit (GE Healthcare). Images were acquired with ImageQuant LAS 4000 (GE Healthcare). The following primary antibodies were used: phosphorylated Src (1:1,000, CST), Src (1:1,000, CST), phosphorylated c-Abl (1:1,000, CST), c-Abl (1:1,000, CST), p62 (1:1,000, MBL), LC-3 (1:1,000, MBL), SOD1 (1:1,000, ENZO), and 13-actin (1:5,000, Sigma).

### Immunoprecipitation

The cells were recovered and lysed in IP buffer containing 1% Triton-X, 0.5% deoxycholate, 50 mM Tris-HCl, 1 mM EDTA, 0.1% SDS, 150 mM NaCl, 0.1% deoxy sodium cholate, protease inhibitor (Roche), and phosphatase inhibitor (Roche). The samples were centrifuged at 13,000×g for 15 min at 4° C. The immunoprecipitation method was performed using immunoprecipitation Kit Dynabeads Protein G (Thermo Fisher Scientific). The supernatant was incubated overnight at 4° C. with protein G conjugated to anti-misfolded SOD1 antibody (MS785) (Fujisawa, T., et al., Ann Neurol 72, 739-749 (2012)). The beads were collected with a magnet, washed 3 times, and then eluted at 70° C. for 10 min. The samples were separated by size on a 10-20% polyacrylamide gel and transferred to Immobilon-P membrane (Millipore). The membrane was blocked with Blocking One (Nacalai Tesque), hybridized with SOD1 antibody (1:1,000, ENZO), and visualized using ECL Prime detection kit (GE Healthcare).

### MN Survival Assay and High Content Analysis

iPS cell cells containing LNI cassette were dissociated into single cells using Accutase, and seeded together with 1 μg/ml of doxycycline (TAKARA, Kusatsu, Japan) in N3 medium containing 1 μM retinoic acid, 1 μM SAG, 10 ng/ml BDNF, 10 ng/ml GDNF, and 10 ng/ml NT-3 on a 96 well plate (BD Bioscience) coated with Matrigel. The cells were fixed and stained on days 7 and 14. The number of viable MNs stained with βIII-tubulin was quantified by IN Cell Analyzer 6000 and expressed as the number of day 14/day 7. Almost 100% βIII-tubulin positive neurons differentiated from iPS cells with LNI cassette showed HB9 positive staining. In this assay, therefore, βIII-tubulin positive neurons were defined as MN. Neurite and cell body were detected by optimized fluorescence levels, and the number of cell bodies having neurite was counted as the number of surviving neurons using IN Cell Developer toolbox software 1.9.

### Large Scale Screen Using ALS MN

iPS cell cells containing LNI cassette were dissociated into single cells using Accutase, and seeded together with 1 ng/ml of doxycycline in N3 medium containing 1 μM RA, 1 μM SAG, 10 ng/ml BDNF, 10 ng/ml GDNF and 10 ng/ml NT-3 on a 96 well plate (BD Bioscience) coated with Matrigel. The libraries used for drug screening were Microsource US Drugs (Microsource Discovery Systems), Microsource International Drugs (Microsource Discovery Systems), and kinase inhibitor purchased from EMD and Selleck Chemicals. Using Integrity and Nextbio data base, existing drugs and test drugs in clinical trials were selected from these libraries and used for throughput screening. On day 7, 1,416 kinds of compounds (final concentration 10 μM) were added, and the cells were fixed and stained on day 14. DMSO was used as a negative control, and kenpaullone, which is a candidate drug for ALS treatment, was used as a positive control. The number of viable MNs stained with βIII-tubulin was quantified by IN Cell Analyzer 6000.

Knock-Down of Src/c-Abl and mTOR Using siRNA in MN

Short interfering RNA (siRNA) was purchased from Life Technologies (siRNA ID of Src siRNA 1: s13414; siRNA ID of Src siRNA 2: s13413; siRNA ID of c-Abl siRNA 1: s866; siRNA ID of c-Abl siRNA 2: s864; siRNA ID of mTOR siRNA: s604). Scramble siRNA purchased from Life Technologies was used as a negative control of siRNA. iPS cells were dissociated and plated with dox in a 96 well plate. On day 7, siRNA was transfected with Lipofectamine RNAiMAX (Thermo Fisher Scientific). Viable MN was evaluated 4 days after transfection by immunostaining with βIII-tubulin, followed by analysis with IN Cell Analyzer 6000 and IN Cell Developer toolbox software 1.9.

### Enzyme-Linked Immunosorbent Assay (ELISA)

Vehicle or 1 μM bosutinib was added to MN on day 7 for 3 days. For ELISA of misfolded SOD1, cells were recovered, and lysed in a buffer containing 1% Triton-X, 0.5% deoxycholate, 50 mM Tris-HCl, 1 mM EDTA, 0.1% SDS, 150 mM NaCl, 0.1% sodium deoxycholate, a protease inhibitor (Roche), and a phosphatase inhibitor (Roche). The samples were centrifuged at 13,000xg for 15 min at 4° C. The 96 well plate (Thermo Fisher Scientific) was coated with 3 μg/ml MS785 antibody at 4° C. in 0.05 M sodium carbonate buffer overnight. After washing and blocking with TBS-T containing 1% BSA, 200 μg of protein was added per 100 μl of sample and the sample was incubated for 2 hr at room temperature. A standard curve was obtained using the recombinant mutant SOD1 protein (G93A). For detection, the plate was incubated together with 3 μg/ml anti-SOD1 antibody (ENZO), and then with sheep anti-rabbit IgG F(ab)′ 2 fragment conjugated with horseradish peroxidase (1:3,000; GE Healthcare). After incubation with tetramethylbenzidine solution (BD Bioscience) for 30 min at room temperature, the absorbance at 450 nm was measured by VersaMax (Molecular Device). For ELISA of p-Src and p-Abl, the cells were collected, and lysed in RIPA buffer containing 0.1% SDS, 150 mM NaCl, 1% NP-40, 0.5% deoxycholate, 50 mM Tris-HCl (pH 8.0), a protease inhibitor (Roche), and a phosphatase inhibitor (Roche). The samples were centrifuged at 13,000×g for 15 min at 4° C. PathScan Phospho-Src (Tyr416) Sandwich ELISA kit (CST), PathScan Phospho-c-Abl (Tyr412) Sandwich ELISA kit (CST), human tyrosine protein kinase src (SRC) ELISA kit (CUSABIO, College Park, MD) human tyrosine protein kinase ABL1 (ABL1) ELISA kit (CUSABIO) and p62 ELISA kit (Enzo) were used according to the protocols of the manufacturers.

Production of Astrocyte from iPS Cells

A minor modification was added to the previously reported method (Kondo, T., et al., Cell Stem Cell 12, 487-496 (2013)), and iPS cells were differentiated into astrocytes. The iPS cells were dissociated into single cells, and re-aggregated in low cell adhesion U-shaped 96-well plates. The aggregates were cultured with DMEM/F12 containing 5% KSR (Thermo Fisher Scientific), 2 μM dorsomorphin (Sigma), 10 μM SB431542 (Cyman Chemical) and 3μ MCHIR99021 (Sigma) for 4 days, and further cultured with 5% KSR, 2 μM dorsomorphin, and 10 μM SB431542 for 10 days. The aggregates were dissociated using Acumax, and cultured in Neurobasal Medium (Thermo Fisher Scientific) and B27 (Thermo Fisher Scientific) on a 24 well plate coated with matrigel. On day 40, the cells were dissociated, seeded on an uncoated 6 well plate, and cultured in DMEM/F12 and 10% FBS (Thermo Fisher Scientific). The passage was repeated every two weeks.

### ATP Measurement

iPS cells were seeded in a 96 well plate at 50,000 cells/well, and MN was produced using N3 medium and dox. On day 7, vehicle and 1 μM bosutinib were added. After 48 hr, the medium was removed from the well, and ATP of the cells was measured using CellTitier-Glo Luminescent Cell Viability Assay (Thermo Fisher Scientific). The ATP level was normalized by the concentration of protein in the cell lysate used for ATP measurement by BCA assay.

### RNA Sequencing of Single Cell

MN on day 7 and labeled with HB9::GFP by lentivirus (Egawa, N., et al., Sci Transl Med 145ra104 (2012)) was isolated using Accumax, and sorted using FACS Ariall (BD Biosciences) on 96 well plates filled with reaction buffer (10 μl) of SMARTer Ultra Low Input RNA-HV kit (Clontech), followed by cDNA synthesis and amplification according to the manufacturer's instructions. The sequencing library was constructed using Nextera XT DNA Sample Prep kit (Illumina). The library was sequenced in 100-cycle Single-Read mode of HiSeq 2500 (Illumina). All sequence reads were extracted in FASTQ format using BCL2FASTQ Conversion Software 1.8.4 in the CASAVA 1.8.4 pipeline. Sequence reads were mapped for the hg19 reference gene and quantified by RPKM for Genes. Biological signatures were estimated using Gene Set Enrichment Analysis (GSEA).

### Dot Blot Analysis

The cells were recovered and lysed in RIPA buffer containing a protease inhibitor and a phosphatase inhibitor. After sonication and centrifugation at 15,000×g for 10 min, each lysed sample (2 μg/spot) was loaded in a nitrocellulose membrane (0.45 μm pore size, GE Healthcare). The membrane was blocked with 5% skim milk, hybridized to an appropriate antibody, and visualized with Western Lightning Plus-ECL (PerkinElmer). Images were acquired with ImageQuant LAS 4000 (GE Healthcare). The following primary antibodies were used: anti-C9orf72 (poly-GP) (1:1,000, Cosmo Bio) and β-actin (1:5,000, Sigma).

### ALS Model Mouse

ALS model mice (B6.Cg-Tg (SOD1*G93A)1Gur/J (Tg-G93A SOD1 mouse)) having G93A mutation and overexpressing human SOD1 gene were obtained from Jackson Laboratories. All animals were cared for, and all procedures were performed according to the guidelines of the Animal Research Institute of Kyoto University. All experiments were approved by the CiRA Animal Care and Use Committee (No.24). From the 8th week after birth, the mice were intraperitoneally injected with 5 mg/kg of bosutinib (Selleck Chemicals) or vehicle (DMSO) 5 times per week for 5 weeks. The onset of the disease was determined to be the time when the body weight reached the maximum, and the final stage was determined to be when the animal placed sideways did not return to the original correct position within 20 seconds (Van Hoecke, A., et al., Nat Med 18, 1418-1422 (2012), Lobsiger, C. S., et al., Proc Natl Acad Sci U S A 110, E4385-4392 (2013)).

### Nissl Staining

Nissl staining and MN counting were performed as described above (Van Hoecke, A., et al., Nat Med 18, 1418-1422 (2012), Lobsiger, C. S., et al., Proc Natl Acad Sci USA 110, E4385-4392 (2013), Fujisawa, T., et al., Hum Mol Genet 25, 245-253 (2016)). Briefly, mice were fixed with 4% PFA, lumbar spinal cord was dehydrated in 30% aqueous sucrose solution and frozen in Tissue-Tek O.T.C. compound (Sakura Finetek). The spinal cord was sliced into 20 μm transverse sections by a cryostat. For Nissl staining, the frozen sections were stained with 1% cresyl violet solution (MP Biomedicals), washed with 100% ethanol and xylene and placed on slides using Mount-Quick Tube (Daido Sangyo). The unilateral anterior horn of the L3 lumbar vertebra was evaluated every 10 sections per animal. Large neurons with a unilated anterior horn diameter greater than 20 μm of the spinal cord were counted.

### Human Post-Mortem Sample

Patients were diagnosed with ALS by El Escorial criteria (Brooks, B. R., et al., Amyotroph Lateral Scler Other Motor Neuron Disord 1, 293-299 (2000)) and pathologically diagnosed by autopsy. The spinal cord was removed and blocks of each level were immediately placed in 10% buffered formalin, embedded in paraffin, and subjected to neuropathological tests or immediately frozen for biochemical tests.

For immunohistochemical tests, control and ALS-derived 6 μm-thick sections fixed in formalin and embedded in paraffin were deparaffinized, antigen-activated by heat/autoclave (121° C., 10 min in 10 mM sodium citrate buffer (pH 6.0)), and then incubated overnight with anti-phosphorylated Src (1:100, R&D) at 4° C. The bound antibody was detected with appropriate Vectastatin Elite ABC kit (Vector Laboratories) using 3,3′-diaminobindidine tetrahydrochloride as the chromogen. Autopsy tissues for immunohistochemistry are detailedly described in Table 3.

For ELISA, frozen spinal block was lysed in RIPA buffer containing 0.1% SDS, 150 mM NaCl, 1% NP-40, 0.5% deoxycholate, 50 mM Tris-HCl (pH8.0), a protease inhibitor (Roche) and a phosphatase inhibitor (Roche). The samples were centrifuged at 13,000×g for 15 min at 4° C.

PathScan Phospho-Src (Tyr416) Sandwich ELISA kit (CST), PathScan Phospho-c-Abl (Tyr412) Sandwich ELISA kit (CST), human tyrosine protein kinase src (SRC) ELISA kit (CUSABIO), and human tyrosine protein kinase ABL1 (ABL1) ELISA kit (CUSABIO) were used according to the manufacturers' protocols. Autopsy tissues for ELISA are detailedly described in Table 4.

TABLE 3
List of autopsy tissues of the spinal cord for immunohistochemistry test

| | | | | | | interval |
|---|---|---|---|---|---|---|
| | | | age at | | | after |
| | sample ID | gender | death (y) | disease | genotype | death (h) |
| control | 189 | male | 75 | meningitis | N.A. | 3.5 |
| control | 5852 | male | 76 | myasthenia | N.A. | 2.9 |
| | | | | gravis | | |
| control | 5922 | male | 60 | myasthenia | N.A. | 2.4 |
| | | | | gravis | | |
| ALS | 5902 | male | 72 | sporadic ALS | N.A. | 2.4 |
| ALS | 5962 | female | 90 | sporadic ALS | N.A. | 9.6 |
| ALS | H283 | male | 62 | sporadic ALS | N.A. | 15.4 |
| ALS | 4330 | female | 40 | familial ALS | SOD1 | 8 |
| | | | | | (L144FVX) | |

TABLE 4
List of autopsy tissues of spinal cord for ELISA

| | | | | | | interval |
|---|---|---|---|---|---|---|
| | | | age at | | | after |
| | sample ID | gender | death (y) | disease | genotype | death (h) |
| control | H270 | female | 75 | dermatomyositis | N.A. | 7.3 |
| control | H290 | male | 61 | dermatomyositis | N.A. | 3.2 |
| control | H315 | male | 71 | multiple system | N.A. | 14 |
| | | | | atrophy | | |
| control | H333 | male | 73 | PSP | N.A. | 2 |
| control | MG1 | male | 76 | myasthenia | N.A. | 2.9 |
| | | | | gravis | | |
| control | MG2 | male | 60 | myasthenia | N.A. | 2.4 |
| | | | | gravis | | |
| control | PSP1 | male | 72 | PSP | N.A. | 7.4 |
| control | PSP2 | male | 68 | PSP | N.A. | 12.2 |
| control | PSP3 | female | 76 | PSP | N.A. | 1.4 |
| control | PSP4 | male | 76 | PSP | N.A. | 23.2 |
| control | MSA1 | male | 66 | multiple system | N.A. | 5.6 |
| | | | | atrophy | | |
| control | PD1 | male | 78 | Parkinson's | N.A. | 1.9 |
| | | | | disease | | |
| ALS | H280 | male | 63 | sporadic ALS | N.A. | 43.2 |
| ALS | H283 | male | 62 | sporadic ALS | N.A. | 15.4 |
| ALS | ALS1 | male | 70 | sporadic ALS | N.A. | 3.5 |
| ALS | ALS3 | male | 63 | sporadic ALS | N.A. | 5.3 |
| ALS | ALS4 | female | 81 | sporadic ALS | N.A. | 6.5 |
| ALS | ALS5 | female | 78 | sporadic ALS | N.A. | 1.4 |
| ALS | ALS6 | male | 55 | sporadic ALS | N.A. | 2.2 |
| ALS | ALS7 | female | 60 | sporadic ALS | N.A. | 9.3 |
| ALS | ALS8 | male | 57 | sporadic ALS | N.A. | 1.6 |

### Statistical Analysis

The results were analyzed using one-way or two-way ANOVA followed by Tukey's post hoc test to determine the statistical significance of the data. The analysis of disease onset and survival period was performed by the log-rank test. Differences were considered significant at p<0.05. The analysis was performed using SPSS software (IBM).

### Results

The MN differentiation method including transducing three transcription factors, LIM homeobox protein 3 (Lhx3), neurogenin 2 (Ngn2), and ISL LIM homeobox 1 (Isl1) into iPS cells was used (see the above-mentioned patent document 5). In summary, the piggyBac vector was used to introduce a polycistronic vector containing Lhx3, Ngn2, and Isl1 under the control of a tetracycline operator into the iPS cells (FIGS. 5A-D and Tables 5 and 6), and clones with introduced vector were established as stable iPS cell clones after neomycin selection.

TABLE 5
List of iPSC clones

| | clone name at | | onset | age at | biopsy | | medication | | |
|---|---|---|---|---|---|---|---|---|---|
| | establishment | gender | age (y) | diagnosis (y) | age (y) | genotype | with riluzole | origin | reprogramming |
| control 1 | 201B7 | female | N.A. | N.A. | 36 | N.A. | N.A. | skin | retrovirus |
| | | | | | | | | fibroblast | |
| control 2 | N112 | female | N.A. | N.A. | 51 | N.A. | N.A. | skin | episomal |
| | | | | | | | | fibroblast | |
| control 3 | N117113 | male | N.A. | N.A. | 74 | N.A. | N.A. | skin | episomal |
| | | | | | | | | fibroblast | |
| control 4 | N116113 | female | N.A. | N.A. | 67 | N.A. | N.A. | skin | episomal |
| | | | | | | | | fibroblast | |
| ALS 1 | A3316 | male | 40 | 40 | 43 | SOD1 | − | skin | episomal |
| (SOD1L144FVX) | | | | | | (L144FVX) | | fibroblast | |
| ALS 2 | A3536 | male | 45 | 45 | 46 | SOD1 | + | skin | episomal |
| (SOD1 L144FVX) | | | | | | (L144FVX) | | fibroblast | |
| ALS 3 | A37228 | female | 29 | 29 | 30 | SOD1 | + | skin | episomal |
| (SOD1 G93S) | | | | | | (G93S) | | fibroblast | |
| ALS 4 | A3411 | female | 55 | 56 | 62 | TDP-43 | + | skin | episomal |
| (TDP-43 M337V) | | | | | | (M337V) | | fibroblast | |
| ALS 5 | A21EL3 | male | 52 | 52 | 55 | TDP-43 | + | peripheral | episomal |
| (TDP-43 Q343R) | | | | | | (Q343R) | | blood mono- | |
| | | | | | | | | nuclear cell | |
| ALS 6 | ND32947E18 | male | 62 | 63 | 64 | TDP-43 | + | skin | episomal |
| (TDP-43 G298S) | | | | | | (G298S) | | fibroblast * | |
| ALS 7 | STI | male | N.R. | N.R. | N.R. | C9orf72repeat | N.R. | skin | episomal |
| (C9orf72) | | | | | | expansion | | fibroblast | |
| ALS 8 | ND06769E4 | female | 45 | 46 | 46 | C9orf72repeat | + | immortalized | episomal |
| (C9orf72) | | | | | | expansion | | B-lymphocyte * | |
| ALS 9 | B463-7 | male | 56 | 64 | 64 | C9orf72repeat | − | skin | sendai virus |
| (C9orf72) | | | | | | expansion | | fibroblast | |
| ALS 10 (SALS) | A13-1 | female | 58 | 59 | 59 | N.A. | + | skin | episomal |
| | | | | | | | | fibroblast | |
| ALS 11 (SALS) | A1142 | male | 64 | 64 | 64 | N.A. | + | skin | retrovirus |
| | | | | | | | | fibroblast | |
| ALS 12 (SALS) | A4114 | male | 30 | 31 | 35 | N.A. | + | skin | episomal |
| | | | | | | | | fibroblast | |

N.A.: Not applicable,
N.R.: not recorded,
* obtained from Coriell Institute

TABLE 6
Mutation in exon region of sporadic ALS-derived iPSC

| ALS genes | ALS10 | ALS11 | ALS12 |
|---|---|---|---|
| SOD1 | none | none | none |
| TDP-43 | none | none | none |

After doxycycline treatment, MNs were produced from iPS cells within 7 days (FIG. 1A). The produced MN expressed MN markers (FIGS. 1B and C), and showed the functional property of MN (FIGS. 1D-F and FIGS. 6A-C).

To establish an ALS-MN phenotypic screening assay, iPSCs were generated from ALS patients with a mutation of L144FVX in SOD1 gene (ALS1) (FIG. 5A-D) and corrected the mutation in the established iPSCs using CRISPR-Cas9 to generate isogenic controls (corrected ALS1) (FIG. 6D-F). iPSCs were differentiated into MNs using transcription factors, and a MN marker, HB9-positive cells were 62.3±2.3% in control, 63.4±1.3% in corrected ALS1-1, and 60.3±2.8% in ALS1 (FIG. 1G and FIG. 6G). In the generated MNs, accumulations of misfolded SOD1 protein (FIGS. 1H and I and FIGS. S2H and I), which plays a pathological role in mutant SOD1-associated ALS was observed [Ann Neurol 72, 739-749 (2012), J Neurochem 113, 1188-1199 (2010)]. Furthermore, vulnerability in ALS-MNs compared with control MNs including mutation-corrected isogenic control (FIGS. 1J and K) was found.

Using this cellular model, the present inventors set up compound screening with a readout of the survival of ALS MNs. iPSCs were differentiated to MNs for 7 days, and chemical compounds were added for another 7 days following evaluation of the surviving MNs by high-content analysis using immunostaining of βIII-tubulin, since nearly 100% of βIII-tubulin-positive neurons expressed HB9 (FIG. 2A and FIGS. 6J and K). Assay performance was determined by calculating the Z′ factor (Z′ factor=0.42±0.30 (mean±SD)). For positive control assays, cells were treated with 50 μM kenpaullone, which was identified as a candidate drug for ALS [Cell Stem Cell 12, 713-726 (2013)], and its positive effect was confirmed; in negative control assays, the cells were treated with vehicle (DMSO). We conducted through-put screening of 1,416 compounds that included drugs both on the market and undergoing clinical trials. The results of the screening are shown in FIG. 2B. Hit compounds were defined as over 3 standard deviations (3SD) from negative controls, and 27 compounds were identified as hits (hit ratio 1.7%) (Table 7).

TABLE 7
List of hit compounds

| hit compounds | MN index |
|---|---|
| tivozanib | 17.25877193 |
| budesonide | 10.26755853 |
| enzastaurin | 9.841029981 |
| VX-680 | 9.57835133 |
| riboflavin | 9.553571429 |
| pazopanib hydrochloride | 9.533464181 |
| alpha-tochopherol | 9.455782313 |
| amodiaquine dihydrochloride | 8.979591837 |
| BMS 777607 | 8.55052879 |
| CYC116 | 8.142394504 |
| sunitinib malate | 8.133120707 |
| bisindolylmaleimide I | 7.963302752 |
| hydroquinone | 7.956989247 |
| flunisolide | 7.857142857 |
| KW 2449 | 7.722412431 |
| MGCD-265 | 7.622860153 |
| PF-2341066 | 7.595240432 |
| hydrastine (1R, 9S) | 7.525083612 |
| piperine | 7.391304348 |
| butamben | 7.391304348 |
| axitinib | 7.352646826 |
| apomorphine hydrochloride | 7.256637168 |
| fenbufen | 7.224080268 |
| bosutinib (SKI-606) | 7.196207532 |
| mebeverine hydrochloride | 6.856187291 |
| flumethasone | 6.755952381 |
| dasatinib | 6.65858685 |

Representative Figures showing the neuroprotective effect of hit drugs are shown in FIG. 2C. We were able to confirm dose-dependency of the protective effect of some hit drugs (FIG. 2D).

Fourteen of the 27 hit compounds were included in the Src/c-Abl-associated pathway (FIG. 2E). Thus, Src/c-Abl as a common target of these hit drugs was focused on. Other Src/c-Abl inhibitors in non-hit drugs of the 1st screening was reevaluated and it was confirmed that they also presented a protective effect, although with lesser efficacy compared with hit drugs (FIG. 7A). Furthermore, knock-down of Src or c-Abl promoted the survival of MNs (FIG. 2F), and the knock-down effects were cancelled by siRNA-resistant forms of Src or c-Abl overexpression (FIG. 7B and C). These results demonstrated the utility of Src and c-Abl pathway as therapeutic targets of ALS-MNs. Among Src/c-Abl inhibitors of the hit drugs, drugs that have direct inhibitory activity for Src/c-Abl, such as bosutinib and dasatinib were focused on. Bosutinib presented dose dependency on MN protection without the bell-shaped responses observed with dasatinib, and the protective effect was exhibited at lower dose compared with other hit drugs in vitro. From these results, bosutinib was selected for further investigation.

We investigated expression and phosphorylation of Src/c-Abl in ALS-MNs. Phosphorylation of Src/c-Abl was increased in mutant SOD1 ALS-MN culture compared with control, and treatment with bosutinib decreased phosphorylation as detected by western blot analysis (FIGS. 2G and H). Typical immunocytochemistry figures of phosphorylated Src (p-Src)/phosphorylated c-Abl (p-c-Abl) are presented in FIG. 2I. Using ELISA, it was also confirmed that phosphorylation of Src/c-Abl was increased in mutant SOD1 ALS-MN culture compared with control, and treatment with bosutinib decreased them (FIG. 2J). Next, the protein level and phosphorylation of Src/c-Abl in other types of cells were evaluated. ALS astrocytes generated from iPSCs (FIG. 8D) and ALS iPSCs exhibited increased phosphorylation of Src without increased phosphorylation of c-Abl (FIG. 8E-H).

To analyze the protective mechanism of bosutinib on ALS-MNs, misfolded protein degradation was investigated. As a result, it was found that p62 levels were elevated in ALS MNs, m which were then reduced by bosutinib treatment, and the change of the LC3-II/LC3-I ratio, suggestive for an autophagic effect in ALS-MN, in ALS-MNs was also attenuated by bosutinib treatment (FIG. 3A-C). To confirm whether the autophagy process was associated with ALS MNs, the effect of the inhibition of mTOR was investigated. The mTOR inhibitor rapamycin, which is known to promote autophagy, and mTOR siRNA increased MN survival (FIG. 3D and E), suggesting that autophagy is impaired in ALS-MNs. Then, to investigate whether the protective effect of bosutinib is associated with the autophagy pathway, autophagy inhibitors, LY294002 and chloroquine, were added to MNs with bosutinib treatment. These autophagy inhibitors partially blocked the protective effect of bosutinib (FIG. 3F). Thus, our data strongly suggested that the protective effect of bosutinib was associated with the promotion of autophagy. Furthermore, it was found that bosutinib treatment reduced the misfolded SOD1 protein levels in ALS-MNs by western blotting (FIG. 3G) and ELISA (FIG. 3H) without decreasing SOD1 mRNA expression levels (FIG. 31). ALS-MN culture also presented a decreasing ATP level, and bosutinib had an attenuating effect on the shortage of intracellular ATP (FIG. 3J). These data suggested that bosutinib promoted the degradation of misfolded SOD1 protein and improved cellular energy shortage. To further explore the molecular background of ALS-MNs, transcriptome analysis was performed using single-cell RNA sequencing (Tables 8 and 9).

TABLE 8-1
Genes highly expressed in mutant SOD1 ALS-MN
identified by single cell RNA-Seq

| Gene name | log2 (fold change) | q-value |
|---|---|---|
| RWDD2B | 14.22 | 0.003 |
| MAD2L1BP | 10.98 | 0.006 |
| DTD1 | 10.77 | 0.001 |
| ZNHIT6 | 9.82 | 0.029 |
| ILF3-AS1 | 9.43 | 0.025 |
| ELP3 | 7.97 | 0.014 |
| CTDSPL2 | 6.81 | 0.039 |
| LINC00665 | 6.61 | 0.019 |
| COMMD9 | 5.14 | 0.005 |
| KIAA1211 | 5.05 | 0.003 |
| ST18 | 4.78 | 0.036 |
| SEC11C | 4.73 | 0.016 |
| RRAGD | 4.19 | 0.013 |
| FBXL20 | 4.12 | 0.047 |
| LIG4 | 3.94 | 0.028 |
| MRPS6/SLC5A3 | 3.50 | 0.013 |
| CLK1 | 3.38 | 0.009 |
| BIRC2 | 3.08 | 0.001 |
| AMY2B/RNPC3 | 3.01 | 0.025 |
| AP1S2 | 2.91 | 0.036 |
| LOC440434 | 2.91 | 0.021 |
| SMIM14 | 2.61 | 0.013 |
| FRYL | 2.60 | 0.009 |
| SELK | 2.60 | 0.005 |
| DCAF6/MPC2 | 2.57 | 0.047 |
| SLU7 | 2.50 | 5.E−04 |
| BEX1 | 2.45 | 0.009 |
| SPTAN1/WDR34 | 2.35 | 0.019 |
| DDX52 | 2.23 | 0.044 |
| OCIAD2 | 2.10 | 3.E−04 |
| SYT11 | 2.05 | 0.028 |
| MAP2 | 2.04 | 0.013 |
| HBS1L | 1.99 | 0.001 |
| SERINCI | 1.94 | 0.004 |
| IP6K2 | 1.77 | 0.014 |
| BEX4 | 1.74 | 0.005 |
| SESN3 | 1.69 | 0.028 |
| RAB3D | 1.67 | 0.009 |
| RWDD1 | 1.67 | 0.006 |
| GFM2/HEXB/NSA2 | 1.65 | 0.044 |
| AUTS2 | 1.65 | 0.038 |
| ZKSCAN1 | 1.62 | 0.009 |

TABLE 8-2
Genes highly expressed in mutant SOD1 ALS-MN
identified by single cell RNA-Seq (continued)

| Gene name | log2 (fold change) | q-value |
|---|---|---|
| FUNDC2 | 1.54 | 0.028 |
| IGFBP7/LOC255130/POLR2B | 1.51 | 0.009 |
| ATP8B5P | 1.51 | 0.040 |
| GDI2 | 1.49 | 0.044 |
| ATP6V0B | 1.49 | 0.004 |
| NDUFA8 | 1.46 | 0.018 |
| PPP2R1A | 1.44 | 0.003 |
| GOLGA4 | 1.41 | 0.038 |
| TMEM245 | 1.34 | 0.044 |
| FNBP1L | 1.34 | 0.040 |
| DNAJA1 | 1.29 | 5.E−05 |
| ZFAND6 | 1.28 | 0.003 |
| GTF2B | 1.28 | 0.038 |
| C11orf57/TIMM8B | 1.22 | 0.013 |
| GPBP1L1 | 1.21 | 0.044 |
| EML4 | 1.16 | 0.038 |
| SCG5 | 1.11 | 0.017 |
| LOC389831 | 1.09 | 0.019 |
| LRRC40 | 1.03 | 0.014 |
| THOC7 | 1.02 | 0.005 |
| POLR2J3 | 1.00 | 0.015 |

Genes with mean expression level of mutant SOD1 ALS-MN more than 2 times higher than that of control MN are listed.

TABLE 9
Genes highly expressed in control MN
identified by single cell RNA-Seq

| Gene name | log2 (fold change) | q-value |
|---|---|---|
| HBG2 | * | 8.7674E−08 |
| HBG1 | * | 8.7674E−08 |
| HBE1 | * | 8.7674E−08 |
| HBA1 | * | 3.9734E−07 |
| ALAS2 | * | 0.0001 |
| HBZ | * | 0.0009 |
| S100A8 | * | 0.0033 |
| TDGF1 | * | 0.0046 |
| AHSP | * | 0.0054 |
| TDGF1P3 | * | 0.0058 |
| AIF1 | * | 0.0172 |
| RHAG | * | 0.0186 |
| CR1L | * | 0.0389 |
| GYPA | * | 0.0448 |
| TAL1 | * | 0.0468 |
| HBA2 | −11.70 | 3.1445E−07 |
| MEG3/MIR770 | −9.32 | 0.0425 |
| MTRNR2L1 | −6.35 | 0.0001 |
| TP53/WRAP53 | −1.70 | 0.0425 |

Genes with mean expression level of control MN more than 2 times higher than that of mutant SOD1 ALS-MN are listed;
* negative infinity

We conducted Gene Set Enrichment Analysis (GSEA) to reveal the biological significance of differentially expressed genes between control and ALS-MNs. As a result, it was found that the increase in mRNA expressions was associated with TCA cycle and respiratory electron transport in ALS-MNs, indicating compensation for energy shortage (FIG. 3K). After treatment with bosutinib, the mRNA expressions associated with TCA cycle and respiratory electron transport decreased in ALS-MNs (FIG. 9).

Furthermore, the effects of Src/c-Abl inhibitor on other genetic types of familial ALS MNs including mutant TDP-43-, C9orf72 repeat expansion-associated familial ALS, and on sporadic ALS were evaluated. Diagnosis of familial ALS was confirmed by genotype (FIG. 5A), and sporadic ALS was examined by re-sequencing using patient fibroblasts (Table 6). TDP-43 inclusions were observed in spinal MNs of a SALS patient (SALS1) by postmortem pathological analysis. MNs were generated from each iPSC (FIG. 4A), and treatment with bosutinib increased surviving MNs in the different types of familial ALS and a part of sporadic ALS (FIG. 4B). Treatment with bosutinib decreased accumulations of abnormal proteins in MNs of familial and sporadic ALS (FIG. 9A-C).

To analyze whether Src/c-Abl inhibitors were effective in vivo, bosutinib was administered to mutant SOD1 transgenic (Tg) mice, a known model for mutant SOD1-asssociated ALS. To investigate the effect of Src/c-Abl inhibitor on MN degeneration in vivo, the same as our in vitro ALS model, treatment with bosutinib (5 mg/kg/day) by intraperitoneal injection was started at age of 8 weeks, and was continued until 13 weeks. Bosutinib delayed disease onset (FIG. 4C) and extended the survival period of mutant SOD1 Tg mice (FIG. 4D). Src/c-Abl were inhibited (FIG. 9D), and misfolded SOD1 proteins in spinal cord were decreased in bosutinib-treated mutant SOD1 Tg mice compared with vehicle treatment (FIG. 4E). The number of MNs was significantly higher in bosutinib-treated mutant SOD1 Tg mice compared with vehicle treatment (FIG. 4F and G). These results indicated that Src/c-Abl inhibition protected MNs from misfolded SOD1-mediated neurodegeneration in vivo.

Finally, the postmortem spinal cord tissue of ALS patients was investigated. Immunoreactivity of phosphorylated Src was increased in the remaining MNs of ALS spinal cords (FIG. 10A, Table 3) as well as that of phosphorylated c-Abl [Katsumata, R., et al., PLos One 7, e46185(2012)], although the trend toward increased phosphorylation of Src in whole ALS spinal cords was not significant (FIG. 10B). Since phosphorylation of Src was increased in ALS patient iPSC-derived MNs, these results suggest that phosphorylation of Src may occur at early stage in ALS, and that patient iPSCs would be useful to analyze ALS patient MNs at early stage before clinical onset.

### INDUSTRIAL APPLICABILITY

The Src/c-Abl pathway inhibitor is useful for the prophylaxis and/or treatment of ALS. Particularly, since clinical and nonclinical data of safety and the like of medicaments already on the market as pharmaceutical products for other diseases have been accumulated, and the libraries of similar compounds already exist, it is expected that a pharmaceutical product capable of preventing and/or treating neurodegenerative diseases can be developed rapidly at a low cost.

This application is based on a patent application No. 2017-226368 filed in Japan (filing date: Nov. 24, 2017), the contents of which are incorporated by reference in full herein.

1.-3. (canceled)

4. A method for preventing and/or treating amyotrophic lateral sclerosis, comprising administering an effective amount of a Src/c-Abl pathway inhibitor to a subject in need of the prevention and/or treatment.

5. A method for screening for a drug for preventing and/or treating amyotrophic lateral sclerosis, comprising contacting Src, c-Abl, a receptor tyrosine kinase that phosphorylates Src or protein kinase C with a test compound, and selecting a test compound that inhibits phosphorylation of the kinase as a candidate for a drug for preventing and/or treating amyotrophic lateral sclerosis.

6. The method according to claim 4, wherein the Src/c-Abl pathway inhibitor is selected from bosutinib, dasatinib, rebastinib, radotinib, tivozanib, pazopanib, sunitinib, BMS777607, CYC116, axitinib, KW2449, VX-680, crizotinib, MGCD-265, enzastaurin, bisindolylmaleimide I, saracatinib, imatinib, nilotinib, and analogs thereof

7. The method according to claim 4, wherein the Src/c-Abl pathway inhibitor is an inhibitory nucleic acid or neutralizing antibody against Src, c-Abl, a receptor tyrosine kinase that phosphorylates Src, or protein kinase C.
